(19) United States
(12) Patent Application Publication
(10) Pub. No.: US 2022/0010020 A1
(43) Pub. Date: Jan. 13, 2022

(54) Anti-FGFR2 Antibody Formulations

(71) Applicant: Five Prime Therapeutics, Inc., South San Francisco, CA (US)

(72) Inventors: Isaias Prado, Vallejo, CA (US); Chin-Yi Huang, Fremont, CA (US)

(73) Assignee: Five Prime Therapeutics, Inc., South San Francisco, CA (US)

(21) Appl. No.: 17/282,589

(22) PCT Filed: Oct. 4, 2019
(86) PCT No.: PCT/US2019/054684

§ 371 Date: Apr. 2, 2021

Related U.S. Application Data
(60) Provisional application No. 62/741,772, filed on Oct. 5, 2018.

(51) Int. Cl. C07K 16/28; A61K 47/26; A61K 47/18

(52) U.S. Cl. CPC C07K 16/2863; A61K 2039/545; A61K 47/183; A61K 47/26

(57) ABSTRACT

This disclosure relates to a formulation of an anti-fibroblast growth factor receptor 2 (FGFR2) antibody that, in some embodiments, is capable of being stored as a liquid, for example in a ready-to-use form, and that, in some embodiments, may be administered intravenously such as by intravenous (IV) infusion.

This application claims the benefit of priority to U.S. Provisional Application No. 62/741,772, which was filed on Oct. 5, 2018, which is incorporated by reference in its entirety.

### TECHNICAL FIELD

This disclosure relates to a formulation for anti-FGFR2 (fibroblast growth factor receptor 2) antibodies, such as anti-FGFR2-IIIb antibodies, that, in some embodiments, is capable of being stored for long periods as a liquid, for example in a ready-to-use form, and that, in some embodiments, may be administered intravenously, such as by intravenous (IV) infusion.

### BACKGROUND AND INTRODUCTION

This disclosure relates to particular formulations of antibodies that bind to FGFR2, e.g. the splice form FGFR2-IIIb (fibroblast growth factor receptor 2 IIIb). FGFR2 is one of four FGFR proteins (FGFR1, 2, 3, and 4). The FGFRs are characterized by multiple alternative splicing of their mRNAs, leading to a variety of isoforms (Ornitz et al., J. Biol. Chem. 271:15292, 1996; see also Swiss-Prot P21802 and isoforms P21802-1 to -20 for sequences of FGFR2 and its isoforms). For FGFR2, alternative splicing leads to isoforms FGFR2-IIIb and FGFR2-IIIc (or just FGFR2b and FGFR2c) for example. The FGFR2-IIIb form of FGFR2 (also denoted K-sam-II) is a high affinity receptor for both FGF1 and KGF family members (FGF7, FGF10, and FGF22), whereas FGFR2-IIIc (also denoted K-sam-I) binds both FGF1 and FGF2 well, but does not bind the KGF family members (Miki et al., Proc. Natl. Acad. Sci. USA 89:246, 1992). FGFR2-IIIb is the only receptor for KGF family members (Ornitz et al., 1996, op. cit.) and is therefore also designated KGFR.

Inhibitors of FGFR2 may include antibodies. For example, U.S. Pat. No. 8,101,723 B2 describes, for example, monoclonal antibodies that bind human FGFR2-IIIb but bind less well or do not bind to FGFR2-IIIc and vice versa. U.S. Patent Publication No. 2015-0050273 A1 describes certain afucosylated antibodies that bind to FGFR2-IIIb. The present disclosure describes formulations that may be used for the antibodies described in U.S. Pat. No. 8,101,723 B2 and U.S. Patent Publication No. 2015-0050273 A1, for example.

Formulation conditions have been identified herein that, in some embodiments, allow the anti-FGFR2 antibodies described herein to be prepared in liquid formulations, remain stable after several months of storage, and be administered intravenously, such as by IV infusion.

### SUMMARY

The present disclosure encompasses pharmaceutical formulations of anti-FGFR2 antibodies, comprising, for example, i) 10-30 mg/mL antibody; ii) 5-40 mM of a buffer selected from one or more of histidine, citrate, or phosphate; iii) 130 to 170 mM arginine or 250-290 mM sucrose; and iv) 0.002% to 0.1% polysorbate 20 or polysorbate 80; wherein the formulation has a pH of 5.0 to 6.5, and wherein the anti-FGFR2 antibody is selected from: a) an antibody comprising a heavy chain comprising the sequence of SEQ ID NO: 2 and a light chain comprising the sequence of SEQ ID NO: 3; b) an antibody comprising a heavy chain comprising a heavy chain (HC) hypervariable region 1 (HVR1) comprising the sequence of SEQ ID NO: 6, an HC HVR2 comprising the sequence of SEQ ID NO: 7, and an HC HVR3 comprising the sequence of SEQ ID NO: 8, and a light chain comprising a light chain (LC) HVR1 comprising the sequence of SEQ ID NO: 9, a LC HVR2 comprising the sequence of SEQ ID NO: 10, and a LC HVR3 comprising the sequence of SEQ ID NO: 11; and c) an antibody comprising a heavy chain comprising a variable region sequence comprising the sequence of SEQ ID NO: 4 and a light chain comprising a light chain variable region sequence comprising the sequence of SEQ ID NO: 5. In some embodiments, the formulation has one or more of the following properties: (a) is a ready-to-use, liquid formulation; (b) is not lyophilized prior to administration to a patient; (c) is contained within a single-use vial; (d) protein aggregation in the formulation increases by no more than 2.0% after 6 months storage at 5° C.; (e) protein aggregation in the formulation increases by no more than 2.0% or 2.5% after 3 months storage at 25° C.; (f) protein aggregation in the formulation increases by no more than 7.0% after 3 months storage at 40° C.; (g) charged protein variants in the formulation do not change by more than 5% after 6 months of storage at 5° C.; (h) protein aggregation in the formulation increases by no more than 2.0% after 5 freeze-thaw cycles at −70° C.; (i) protein aggregation in the formulation increases by no more than 2.0% after 72 hours of mechanical stress at 500 rpm; (j) is diluted in saline solution prior to intravenous administration; (k) is administered intravenously (e.g. by intravenous infusion); and (1) is isotonic with human plasma.

In some cases, the above formulations comprise 10-15 mg/mL, 15-20 mg/mL, 20-25 mg/mL, 18-22 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, or 25 mg/mL of the anti-FGFR2 antibody. In some cases, the formulations comprise 10-40 mM, 10-30 mM, 15-25 mM, 10-20 mM, 20-30 mM, 18-22 mM, 10 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, or 30 mM of the buffer. In some embodiments, the buffer is a histidine buffer. In some embodiments, the formulation comprises 130-150 mM, 150-170 mM, 140-160 mM, 130 mM, 135 mM, 140 mM, 145 mM, 150 mM, 155 mM, 160 mM, 165 mM, or 170 mM arginine. In some cases, a formulation comprising arginine does not comprise sucrose. In some embodiments, the pH of an arginine-comprising formulation is 5.0-7.0, 5.0-6.0, 5.5-6.0, 5.5-6.5, 5.5-5.9, 5.6-5.8, 5.0, 5.1, 5.2, 5.3, 5.4, 5.5, 5.6, 5.7, 5.8, 5.9, 6.0, 6.1, 6.2, 6.3, 6.4, or 6.5. In some cases, the pH of the formulation is 5.5-5.9, 5.6-5.8, 5.6, 5.7, 5.8, 5.9, or 6.0. In some embodiments, the formulation comprises 260-280 mM, 250 mM, 260 mM, 270 mM, 280 mM, 290 mM sucrose. In some such cases, the formulation comprising sucrose does not comprise arginine. In some embodiments, the pH of a sucrose-comprising formulation is 5.0-7.0, 5.0-6.0, 5.5-6.0, 5.5-5.9, 5.6-5.8, 5.0, 5.1, 5.2, 5.3, 5.4, 5.5, 5.6, 5.7, 5.8, 5.9, 6.0, 6.1, 6.2, 6.3, 6.4, or 6.5. In some embodiments, the pH of the formulation is 5.8-6.2, 5.9-6.1, 5.9, 6.0, or 6.1.

Any of the above formulations may also comprise 0.01-0.1%, 0.005-0.05%, 0.002%, 0.003%. 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, or 0.1% polysorbate 20 or 80. In some cases, the formulations comprise 0.01-0.1%, 0.005-0.05%, 0.002%, 0.003%. 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, or 0.1% polysorbate 20.

In some embodiments, the formulations consist essentially of anti-FGFR2 antibody; citrate, phosphate, or histidine buffer; arginine or sucrose; and polysorbate 20 or 80. In some such cases, the formulations consist essentially of anti-FGFR2 antibody, histidine, arginine or sucrose, and polysorbate 20.

The present disclosure also includes, for instance, a pharmaceutical formulation of an anti-FGFR2 antibody, wherein the formulation is a liquid formulation that has not been lyophilized prior to use, comprising: i) 10-25 mg/mL of the anti-FGFR2 antibody; ii) 10-30 mM histidine buffer; iii) 140-160 mM arginine; and iv) 0.005% to 0.05% polysorbate 20; wherein the formulation has a pH of 5.6 to 5.8. The present disclosure also includes, for instance, a pharmaceutical formulation of an anti-FGFR2 antibody, wherein the formulation is a liquid formulation that has not been lyophilized prior to use, comprising: i) 10-25 mg/mL of the anti-FGFR2 antibody; ii) 20 mM histidine buffer; iii) 150 mM arginine; and iv) 0.01% polysorbate 20; wherein the formulation has a pH of 5.7. Embodiments herein further include pharmaceutical formulations of an anti-Fibroblast Growth Factor Receptor 2 (FGFR2) antibody, comprising: i) 20 mg/mL of the anti-FGFR2 antibody; ii) 20 mM L-histidine; iii) 150 mM L-arginine; iv) 0.01% polysorbate 20, wherein the pharmaceutical formulation has a pH of 5.7 and is a liquid formulation that has not been lyophilized prior to use. In any of these cases, in some embodiments, the formulation consists essentially of the anti-FGFR2 antibody, histidine, arginine, and polysorbate 20. In some such embodiments, the anti-FGFR2 antibody may be selected from: a) an antibody comprising a heavy chain comprising the sequence of SEQ ID NO: 2 and a light chain comprising the sequence of SEQ ID NO: 3; b) an antibody comprising a heavy chain comprising a heavy chain (HC) hypervariable region 1 (HVR1) comprising the sequence of SEQ ID NO: 6, an HC HVR2 comprising the sequence of SEQ ID NO: 7, and an HC HVR3 comprising the sequence of SEQ ID NO: 8, and a light chain comprising a light chain (LC) HVR1 comprising the sequence of SEQ ID NO: 9, a LC HVR2 comprising the sequence of SEQ ID NO: 10, and a LC HVR3 comprising the sequence of SEQ ID NO: 11; and c) an antibody comprising a heavy chain comprising a variable region sequence comprising the sequence of SEQ ID NO: 4 and a light chain comprising a light chain variable region sequence comprising the sequence of SEQ ID NO: 5. The pharmaceutical formulations described above may have one or more of the following properties: (a) is contained within a single-use vial; (b) protein aggregation in the formulation increases by no more than 2.0% after 6 months storage at 5° C.; (c) protein aggregation in the formulation increases by no more than 2.0% or 2.5% after 3 months storage at 25° C.; (d) protein aggregation in the formulation increases by no more than 7.0% after 3 months storage at 40° C.; (e) charged protein variants in the formulation do not change by more than 5% after 6 months of storage at 5° C.; (f) protein aggregation in the formulation increases by no more than 2.0% after 5 freeze-thaw cycles at −70° C.; (g) protein aggregation in the formulation increases by no more than 2.0% after 72 hours of mechanical stress at 500 rpm; (h) is diluted in saline solution prior to intravenous administration; (i) is administered intravenously (e.g. by IV infusion); and (j) is isotonic with human plasma. The pharmaceutical formulation above that comprise 10-25 mg/mL antibody may, more specifically, comprise 10-15 mg/mL, 15-20 mg/mL, 20-25 mg/mL, 18-22 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, or 25 mg/mL of the anti-FGFR2 antibody.

The present disclosure also includes, for instance, a pharmaceutical formulation of an anti-FGFR2 antibody, wherein the formulation is a liquid formulation that has not been lyophilized prior to use, comprising: i) 10-25 mg/mL of the anti-FGFR2 antibody; ii) 10-30 mM histidine buffer; iii) 260-280 mM sucrose; and iv) 0.005% to 0.05% polysorbate 20; wherein the formulation has a pH of 5.8 to 6.2. The present disclosure also includes, for instance, a pharmaceutical formulation of an anti-FGFR2 antibody, wherein the formulation is a liquid formulation that has not been lyophilized prior to use, comprising: i) 10-25 mg/mL of the anti-FGFR2 antibody; ii) 20 mM histidine buffer; iii) 270 mM sucrose; and iv) 0.01% polysorbate 20; wherein the formulation has a pH of 6.0. In further embodiments, the pharmaceutical formulation may comprise: i) 20 mg/mL of the anti-FGFR2 antibody; ii) 20 mM L-histidine; iii) 270 mM sucrose; and iv) 0.01% polysorbate 20, wherein the formulation has a pH of 6.0 and is a liquid formulation that has not been lyophilized prior to use. In some such embodiments, the formulation consists essentially of the anti-FGFR2 antibody, histidine, sucrose, and polysorbate 20. In some such embodiments, the anti-FGFR2 antibody may be selected from: a) an antibody comprising a heavy chain comprising the sequence of SEQ ID NO: 2 and a light chain comprising the sequence of SEQ ID NO: 3; b) an antibody comprising a heavy chain comprising a heavy chain (HC) hypervariable region 1 (HVR1) comprising the sequence of SEQ ID NO: 6, an HC HVR2 comprising the sequence of SEQ ID NO: 7, and an HC HVR3 comprising the sequence of SEQ ID NO: 8, and a light chain comprising a light chain (LC) HVR1 comprising the sequence of SEQ ID NO: 9, a LC HVR2 comprising the sequence of SEQ ID NO: 10, and a LC HVR3 comprising the sequence of SEQ ID NO: 11; and c) an antibody comprising a heavy chain comprising a variable region sequence comprising the sequence of SEQ ID NO: 4 and a light chain comprising a light chain variable region sequence comprising the sequence of SEQ ID NO: 5. The pharmaceutical formulations described above may have one or more of the following properties: (a) is contained within a single-use vial; (b) protein aggregation in the formulation increases by no more than 2.0% after 6 months storage at 5° C.; (c) protein aggregation in the formulation increases by no more than 2.0% or 2.5% after 3 months storage at 25° C.; (d) protein aggregation in the formulation increases by no more than 7.0% after 3 months storage at 40° C.; (e) charged protein variants in the formulation do not change by more than 5% after 6 months of storage at 5° C.; (f) protein aggregation in the formulation increases by no more than 2.0% after 5 freeze-thaw cycles at −70° C.; (g) protein aggregation in the formulation increases by no more than 2.0% after 72 hours of mechanical stress at 500 rpm; (h) is diluted in saline solution prior to intravenous administration; (i) is administered intravenously (e.g. by IV infusion); and (j) is isotonic with human plasma. The pharmaceutical formulation above that comprise 10-25 mg/mL antibody may, more specifically, comprise 10-15 mg/mL, 15-20 mg/mL, 20-25 mg/mL, 18-22 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, or 25 mg/mL of the anti-FGFR2 antibody.

In some embodiments of this disclosure, the pharmaceutical formulation may not comprise one or more of the following: sugars other than sucrose, sugar alcohols, protein species other than anti-FGFR2 antibody, surfactants other than polysorbate 20 or polysorbate 80, amino acids other than arginine and histidine, Cu²⁺, Mg²⁺, and Mn²⁺. In any embodiments of this disclosure, the formulation may be contained within single-use vials.

The present disclosure also encompasses methods of treating a solid tumor in a patient in need thereof, comprising administering an effective amount of pharmaceutical formulation herein to a patient. In some embodiments, the formulation is administered intravenously to the patient (e.g., by intravenous infusion).

In any of the formulations herein, the anti-FGFR2 antibody can be optionally afucosylated. In any of the formulations herein, the anti-FGFR2 antibody can be a full length antibody, or can be an antigen binding fragment, such as an Fv, single-chain Fv (scFv), Fab, Fab′, or (Fab′)₂. In any of the formulations herein, the antibody can be chimeric, humanized, or human. In any of the formulations herein, the antibody can be bispecific, multispecific, or conjugated.

### BRIEF DESCRIPTION OF THE FIGURES

Figure (FIG. 1 shows average binding of an afucosylated anti-FGFR2IIIb antibody to FGFR2IIIb or to protein A in a Biacore® assay after incubation of the antibody in different percent concentrations of hydrogen peroxide (0.01%, 0.1%, and 1.0% as well as 0% (control)). Incubation of the anti-FGFR2IIIb antibody with hydrogen peroxide did not interfere with binding to FGFR2IIIb, although it did interfere to some extent with protein A binding.

FIG. 2 shows differential scanning calorimetry (DSC) thermograms of citrate/NaCl formulations of anti-FGFR2IIIb antibody at pH 4.0, 5.0 and 6.0. The data show that the unfolding temperature of the formulations increases with pH in this range.

FIG. 3 shows size exclusion high performance liquid chromatography SE-HPLC chromatograms of anti-FGFR2IIIb antibody screening samples at pH 4, 5, 6, 7, and 8, as discussed in Example 5 below.

FIG. 4 shows impact of buffer pH (in citrate or phosphate NaCl buffer) on aggregate formation as determined by SE-HPLC in a range of pH 4-8 and over a storage period of 0-2 months at 40° C. The figure shows a large % increase in aggregates at pH 4 compared to the other pHs.

FIG. 5 shows impact of buffer pH (in citrate or phosphate NaCl buffer) on clip (fragment) formation as determined by SE-HPLC in a range of pH 4-8 and over a storage period of 0-2 months at 40° C. The figure shows a large % increase in clip formation at pH 4 compared to the other pHs.

FIG. 6 shows impact of buffer pH (in citrate or phosphate NaCl buffer) on aggregate formation as determined by SE-HPLC in a range of pH 4-8 and over a storage period of 0-2 months at 25° C. The figure shows that all of the formulations comprised generally less than 3.0% aggregates over this time period except for the pH 8 formulation, for which aggregates increased from about 2.5% to about 4% over the two months of storage.

FIG. 7 shows impact of buffer pH (in citrate or phosphate NaCl buffer) on clip (fragment) formation as determined by SE-HPLC in a range of pH 4-8 and over a storage period of 0-2 months at 25° C. The figure shows an increase to nearly 1.5% in clips at pH 4 compared to the other pHs, which remain below 0.5% clips.

FIG. 8 shows a representative weak cation exchange (WCX)-HPLC chromatogram of an anti-FGFR2IIIb formulation to evaluate the charge variant profile.

FIG. 9 shows impact of buffer pH (in citrate or phosphate NaCl buffer) on acidic variant formation as determined by WCX-HPLC in a range of pH 4-8 and over a storage period of 0-2 months at 25° C. There was a relatively large % increase in acidic variants in the pH 8 formulation, with the pH 7 formulation showing the next largest % increase.

FIG. 10 shows impact of buffer pH (in citrate or phosphate NaCl buffer) on basic variant formation as determined by WCX-HPLC in a range of pH 4-8 and over a storage period of 0-2 months at 25° C. There was a relatively large % decrease in basic variants in the pH 8 formulation, with the pH 7 formulation showing the next largest % decrease. The smallest % change was observed at pH 4.

FIG. 11 shows impact of buffer pH (in citrate or phosphate NaCl buffer) on the size of the main peak in the WCX-HPLC chromatogram in a range of pH 4-8 and over a storage period of 0-2 months at 25° C. The largest % changes in the main peak were observed in the pH 4 and pH 8 formulations and the smallest % changes in the pH 5 and pH 6 and pH 7 formulations.

FIG. 12 shows the solution appearance of seven anti-FGFR2IIIb formulations shown in Table 8 below after shaking at 500 rpm for 72 hours.

FIG. 13 shows the impact of excipients on anti-FGFR2IIIb stability against 5 freeze-thaw cycles at 70° C. to ambient temperature. Formulations are as provided in Table 8. Only the histidine/NaCl formulation (no. 4 in Table 8) appeared to show an increase in aggregates upon the multiple freeze-thaw cycles.

FIG. 14 shows the impact of buffer and bulking agent on anti-FGFR2IIIb aggregate formation over 1 month at 40° C. as determined by SE-HPLC.

FIG. 15 shows the impact of buffer and bulking agent on anti-FGFR2IIIb aggregate formation over 3 months at 40° C. as determined by SE-HPLC.

FIG. 16 shows the impact of buffer and bulking agent on anti-FGFR2IIIb acidic variant formation over 3 months at 25° C. as determined by WCX-HPLC.

FIG. 17 shows the impact of buffer and bulking agent on the size of the main WCX-HPLC peak over 3 months at 25° C.

FIG. 18 shows the impact of buffer and bulking agent on the size of the main WCX-HPLC peak over 3 months at 25° C.

FIG. 19 shows the impact of mechanical stress over 72 hours on histidine/arginine or histidine/sucrose anti-FGFR2IIIb antibody formulations at pH 5.5 to 6.5 as determined by analysis of aggregates by SE-HPLC.

FIG. 20 shows the impact of mechanical stress over 72 hours on histidine/arginine or histidine/sucrose anti-FGFR2IIIb antibody formulations at pH 5.5 to 6.5 as determined by analysis of clips (fragments) by SE-HPLC.

FIG. 21 shows the impact of 5 cycles of freeze-thaw at −70° C. to ambient temperature on histidine/arginine or histidine/sucrose anti-FGFR2IIIb antibody formulations at pH 5.5 to 6.5 as determined by analysis of aggregates by SE-HPLC.

FIG. 22 shows the impact of 5 cycles of freeze-thaw at −70° C. to ambient temperature on histidine/arginine or histidine/sucrose anti-FGFR2IIIb antibody formulations at pH 5.5 to 6.5 as determined by analysis of clips (fragments) by SE-HPLC.

FIGS. 23A-C show the effect of pH between pH 5.5 and 6.5 on aggregate formation in a histidine/arginine formulation comprising 20 mM histidine, 150 mM arginine, and 0.01% polysorbate 20. FIG. 23A shows aggregate formation, indicating that, at pH 5.5-6.0, after 1 month at 40° C., % aggregates remained generally below 3%. FIG. 23B shows aggregate formation, indicating that, at pH 5.5-6.0, after 3 months at 25° C., % aggregates remained generally below 2.5%. FIG. 23C shows aggregate formation, indicating that, at all pHs, after 6 months at 5° C., % aggregates remained generally about 1.5% and less than 2%.

FIGS. 24A-C show the effect of pH between pH 5.5 and 6.5 on clips (fragments) in a histidine/arginine formulation comprising 20 mM histidine, 150 mM arginine, and 0.01% polysorbate 20. FIG. 24A shows clip (fragment) formation, indicating that, at all pHs, after 1 month at 40° C., % clips remained generally about 1% or less than 1% at pH 6. FIG. 24B shows clips formation, indicating that, at all pHs, after 3 months at 25° C., % clips remained generally below 1.5%. FIG. 24C shows clips formation, indicating that, at all pHs, after 6 months at 5° C., % clips remained generally less than 1%, with % clips in formulations at pH 5.7 or 6.0 remaining generally below 0.5%.

FIGS. 25A-C show the effect of pH between pH 5.5 and 6.5 on aggregate formation in a histidine/sucrose formulation comprising 20 mM histidine, 270 mM sucrose, and 0.01% polysorbate 20. FIG. 25A shows aggregate formation, indicating that, at pH 5.5-6.0, after 1 month at 40° C., % aggregates remains generally below 4%, and are below 3% at pH 6.0 and 6.3 and about 2.5% at pH 5.5 and 5.7. FIG. 25B shows aggregate formation, indicating that, at pH 5.5-5.7, after 3 months at 25° C., % aggregates remains generally below 2.0% and that at pH 6.0, % aggregates remains below 2.5%. FIG. 25C shows aggregate formation, indicating that, at all pHs, after 6 months at 5° C., % aggregates remained generally about 1.5% and less than 2%.

FIGS. 26A-C show the effect of pH between pH 5.5 and 6.5 on clips (fragments) in a histidine/sucrose formulation comprising 20 mM histidine, 270 mM sucrose, and 0.01% polysorbate 20. FIG. 26A shows clip (fragment) formation, indicating that, at all pHs, after 1 month at 40° C., % clips remained generally less than 2% or less than 1.5% at pH 5.5 to 6.3. FIG. 26B shows clip formation, indicating that, at all pHs, after 3 months at 25° C., % clips remained generally below 3.5%, but that clip formation is pH dependent with the lowest percentage at 1, 2, and 3 months for the pH 5.5 formulation and the highest percentage at each month for the pH 6.3 formulation. FIG. 26C shows clips formation, indicating that, at pHs of 6.0 or below, after 6 months at 5° C., % clips remains generally less than 1%.

FIGS. 27A-C show effect of pH between pH 5.5 and 6.5 on acidic variant formation in a histidine/sucrose formulation comprising 20 mM histidine, 270 mM sucrose, and 0.01% polysorbate 20. FIG. 27A shows acidic variant formation, indicating that, at pH 5.5-6.0, after 1 month at 40° C., % acidic variants remained generally below 40%. FIG. 27B shows acidic variant formation, indicating that, at pH 5.5-6.0, after 3 months at 25° C., % acidic variants remained generally below 30.0%. FIG. 27C shows acidic variant formation, indicating that, at all pHs, after 6 months at 5° C., % acidic variants remained generally below 25%.

FIGS. 28A-C show effect of pH between pH 5.5 and 6.5 on acidic variant formation in a histidine/arginine formulation comprising 20 mM histidine, 150 mM arginine, and 0.01% polysorbate 20. FIG. 28A shows acidic variant formation, indicating that, at pH 5.5-6.0, after 1 month at 40° C., % acidic variants remained generally below 40%. FIG. 28B shows acidic variant formation, indicating that, at pH 5.5-6.0, after 3 months at 25° C., % acidic variants remained generally below 30.0%. FIG. 28C shows acidic variant formation, indicating that, at all pHs, after 6 months at 5° C., % acidic variants remained generally below 25%.

FIGS. 29A-C show effect of pH between pH 5.5 and 6.5 on basic variant formation in a histidine/sucrose formulation comprising 20 mM histidine, 270 mM sucrose, and 0.01% polysorbate 20. FIG. 29A shows basic variant formation, indicating that, at pH 5.5-6.0, after 1 month at 40° C., % basic variants remained mostly between 10% and 20%.

FIG. 29B shows basic variant formation, indicating that, at pH 5.5-6.0, after 3 months at 25° C., % basic variants remained generally between 20% and 30%. FIG. 29C shows basic variant formation, indicating that, at all pHs, after 6 months at 5° C., % basic variants remained generally between 25% and 30%.

FIGS. 30A-C show effect of pH between pH 5.5 and 6.5 on basic variant formation in a histidine/arginine formulation comprising 20 mM histidine, 150 mM arginine, and 0.01% polysorbate 20. FIG. 30A shows basic variant formation, indicating that, at pH 5.5-6.0, after 1 month at 40° C., % basic variants remained mostly between 10% and 20%. FIG. 30B shows basic variant formation, indicating that, at pH 5.5-6.0, after 3 months at 25° C., % basic variants remained generally between 15% and 25%. FIG. 30C shows basic variant formation, indicating that, at all pHs, after 6 months at 5° C., % basic variants remained generally between 25% and 30%.

See the Examples section herein for further details on the above figures.

### DETAILED DESCRIPTION

The section headings used herein are for organizational purposes only and are not to be construed as limiting the subject matter described. All references cited herein, including patent applications and publications, are incorporated herein by reference in their entireties for any purpose.

### Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

In this application, the use of “or” means “and/or” unless stated otherwise. In the context of a multiple dependent claim, the use of “or” refers back to more than one preceding independent or dependent claim in the alternative only. Also, terms such as “element” or “component” encompass both elements and components comprising one unit and elements and components that comprise more than one subunit unless specifically stated otherwise.

As described herein, any concentration range, percentage range, ratio range or integer range is to be understood to include the value of any integer within the recited range and, when appropriate, fractions thereof (such as one tenth and one hundredth of an integer), unless otherwise indicated.

Units, prefixes, and symbols are denoted in their Système International de Unites (SI) accepted form. Numeric ranges are inclusive of the numbers defining the range. Measured values are understood to be approximate, taking into account significant digits and the error associated with the measurement.

The headings provided herein are not limitations of the various aspects of the disclosure, which can be had by reference to the specification as a whole. Accordingly, the terms defined immediately below are more fully defined by reference to the specification in its entirety.

As utilized in accordance with the present disclosure, the following terms, unless otherwise indicated, shall be understood to have the following meanings:

“Administering” refers to the physical introduction of a composition comprising a therapeutic agent to a subject, using any of the various methods and delivery systems known to those skilled in the art. General routes of administration for antibodies include intravenous (IV), intramuscular, subcutaneous (SC), intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Non-parenteral routes include a topical, epidermal or mucosal route of administration, for example, orally, intranasally, vaginally, rectally, sublingually or topically. Administering can also be performed, for example, once, a plurality of times, and/or over one or more extended periods.

A “pharmaceutical formulation” or “therapeutic formulation” as used herein refers to a composition comprising a therapeutic agent such as an antibody that is suitable for pharmaceutical use, such as, for example, administration to a patient either directly or after being reconstituted, diluted, mixed, or dissolved with at least one further composition, or thawed from a frozen state. A “ready-to-use” formulation, as used herein, means a formulation that can be administered to a patient directly and therefore, does not need to be diluted, reconstituted, thawed from a frozen state, dissolved in solution, or mixed with other ingredients prior to administration.

As used herein, “subcutaneous administration” refers to administration of a therapeutic underneath the skin. In some cases, subcutaneous administration (abbreviated SC or SubQ) can be performed using a needle connected to a syringe or pump or with a special type of injector device (e.g. EpiPen® and the like). In some cases, SC administration may release the therapeutic into the tissue between the skin and muscle layers, and in some cases into the muscle layers.

As used herein, an “injectable” formulation is one in which is suitable for administration by SC injection.

As used herein, an “intravenous administration” refers to administration of a therapeutic directly into a vein of a subject. Intravenous administration may be by “intravenous infusion.” Infusion refers to the use of pressure supplied by gravity to deliver the therapy into the patient.

Formulations herein may be contained in “vials.” A “vial” herein refers to any small container suitable for holding a liquid pharmaceutical formulation. In some cases, a “vial” may be connected to a syringe or pump or the “vial” may be a part of an administration device, for example, for injecting the contents of the vial into the patient. In some embodiments, formulations may be comprised in “single-use vials.” A “single-use vial” refers to a “vial” in which, after the contents have been administered, the entire vial or the remaining contents of the vial are discarded.

The term “lyophilized” when applied to a formulation, material, or composition refers to a freeze-dried formulation, material, or composition.

As used herein, the term “liquid formulation” means a formulation that is in the liquid state, including, for example, a solution or liquid emulsion.

A “buffer” as used herein is a substance that, when added to a solution or formulation, resists significant changes in pH upon dilution or upon addition of acidic or basic components. Exemplary buffers herein include histidine, citrate, and phosphate buffers.

An “isotonic” formulation, as referred to herein, is a formulation that has an osmotic pressure that is about equivalent to that of human bodily fluids into which the formulation is introduced or that reside in the area of administration, such as human blood plasma or human lymphatic fluid.

The “aggregation” or “protein aggregation” in a formulation, as used herein, refers to aggregation of polypeptide molecules in the formulation. The amount of polypeptides in a formulation that are in an aggregated state, such as in dimers or multimers, may be expressed, for example, as a percentage of the total polypeptide content of the formulation. Aggregation may be detected, for example, by size exclusion chromatography or other techniques that separate proteins in a solution based upon size or molecular weight. In such techniques, the amount of “aggregates” may be equivalent to the amount of polypeptides in a formulation that have at least twice the molecular weight as the main polypeptide species of the formulation (e.g. an anti-FGFR2-IIIb antibody). Species with twice the molecular weight as the main polypeptide species of the formulation may be considered “dimers” herein, while detectable species with molecular weights larger than that of dimers may be termed “higher molecular weight aggregates” or “multimers” or a like term.

The phrase “in the dark,” when referring to storage of a formulation herein, means that the formulation is stored such that it is protected from exposure to ambient and ultraviolet light. For example, the formulation may be stored in a dark room or space, or within packaging that protects the formulation against such light, or within a vial with walls are made of material or covered with material that protects the contents against such light.

In some embodiments herein, pharmaceutical formulations “do not comprise” one or more types of excipients or ingredients. The expression “does not comprise” in this context means that the excluded ingredients are not present beyond trace levels, for example, due to contamination or impurities found in other purposefully added ingredients.

The term “consisting essentially of” when referring to a mixture of ingredients of a formulation herein indicates that, while ingredients other than those expressly listed may be present, such ingredients are found only in trace amounts or in amounts otherwise low enough that the fundamental characteristics of the formulation including protein concentration, viscosity, thermal stability, osmolality, and pH are unchanged.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

“FGFR2” refers to human fibroblast growth factor receptor 2 including any of its alternatively spliced forms such as the Ma, IIIb and Mc splice forms, unless a non-human FGFR2 is explicitly indicated (e.g. “murine FGFR2”). The term FGFR2 encompasses wild-type FGFR2 and naturally occurring mutant forms such as FGFR2 activating mutant forms such as FGFR2-S252W, which is found in some cancer cells. “FGFR2-IIIb” or “FGFR2b” are used interchangeably to refer to the fibroblast growth factor receptor 2 IIIb splice form. An exemplary human FGFR2-IIIb is shown in GenBank Accession No. NP_075259.4, dated Jul. 7, 2013. A nonlimiting exemplary mature human FGFR2-IIIb amino acid sequence is shown in SEQ ID NO: 1. “FGFR2-IIIc” or “FGFR2c” are used interchangeably to refer to the fibroblast growth factor receptor 2 IIIc splice form. An exemplary human FGFR2-IIIc is shown in GenBank Accession No. NP_000132.3, dated Jul. 7, 2013. A nonlimiting exemplary mature FGFR2-IIIc amino acid sequence is shown in SEQ ID NO: 13.

With reference to anti-FGFR2-IIIb antibodies, the terms “blocks binding od” or “inhibits binding of” a ligand refer to the ability to inhibit an interaction between FGFR2-IIIb and an FGFR2 ligand, such as FGF1 or FGF2. Such inhibition may occur through any mechanism, including direct interference with ligand binding, e.g., because of overlapping binding sites on FGFR2-IIIb, and/or conformational changes in FGFR2-IIIb induced by an antibody that alter ligand affinity.

“Affinity” or “binding affinity” refers to the strength of the sum total of noncovalent interactions between a single binding site of a molecule (e.g., an antibody) and its binding partner (e.g., an antigen). In some embodiments, “binding affinity” refers to intrinsic binding affinity which reflects a 1:1 interaction between members of a binding pair (e.g., antibody and antigen). The affinity of a molecule X for its partner Y can generally be represented by the dissociation constant (K_{d}).

The term “antibody” as used herein refers to a molecule comprising at least hypervariable regions (HVRs) H1, H2, and H3 of a heavy chain and L1, L2, and L3 of a light chain, wherein the molecule is capable of binding to antigen. The term antibody includes, but is not limited to, antibodies comprising full length heavy and light chains as well as fragments that are capable of binding antigen (also called “antigen-binding fragments” herein), such as Fv, single-chain Fv (scFv), Fab, Fab′, and (Fab′)₂. The term antibody also includes, but is not limited to, chimeric antibodies, humanized antibodies, human antibodies, and antibodies of various species such as mouse, human, cynomolgus monkey, etc. It also includes antibodies conjugated to other molecules such as small molecule drugs, bispecific antibodies and multispecific antibodies.

The term “heavy chain variable region” refers to a region comprising heavy chain HVR1, framework (FR) 2, HVR2, FR3, and HVR3. In some embodiments, a heavy chain variable region also comprises at least a portion of an FR1 and/or at least a portion of an FR4.

The term “heavy chain constant region” refers to a region comprising at least three heavy chain constant domains, C_{H}1, C_{H}2, and C_{H}3. Nonlimiting exemplary heavy chain constant regions include γ, δ, and α. Nonlimiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an a constant region is an IgA antibody. Further, an antibody comprising a μ constant region is an IgM antibody, and an antibody comprising an ε constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

The term “heavy chain” refers to a polypeptide comprising at least a heavy chain variable region, with or without a leader sequence. In some embodiments, a heavy chain comprises at least a portion of a heavy chain constant region. The term “full-length heavy chain” refers to a polypeptide comprising a heavy chain variable region and a heavy chain constant region, with or without a leader sequence.

The term “light chain variable region” refers to a region comprising light chain HVR1, framework (FR) 2, HVR2, FR3, and HVR3. In some embodiments, a light chain variable region also comprises an FR1 and/or an FR4.

The term “light chain constant region” refers to a region comprising a light chain constant domain, C_{L}. Nonlimiting exemplary light chain constant regions include λ and κ.

The term “light chain” refers to a polypeptide comprising at least a light chain variable region, with or without a leader sequence. In some embodiments, a light chain comprises at least a portion of a light chain constant region. The term “full-length light chain” refers to a polypeptide comprising a light chain variable region and a light chain constant region, with or without a leader sequence.

The term “hypervariable region” or “HVR” refers to each of the regions of an antibody variable domain that are hypervariable in sequence and/or form structurally defined loops (“hypervariable loops”). Generally, native four-chain antibodies comprise six HVRs; three in the V_{H} (H1, H2, H3), and three in the V_{L} (L1, L2, L3). HVRs generally comprise amino acid residues from the hypervariable loops and/or from the “complementarity determining regions” (CDRs), the latter being of highest sequence variability and/or involved in antigen recognition. Exemplary hypervariable loops occur at amino acid residues 26-32 (L1), 50-52 (L2), 91-96 (L3), 26-32 (H1), 53-55 (H2), and 96-101(H3). (Chothia and Lesk, J. Mol. Biol. 196:901-917 (1987).) Exemplary CDRs (CDR-L1, CDR-L2, CDR-L3, CDR-H1, CDR-H2, and CDR-H3) occur at amino acid residues 24-34 of L1, 50-56 of L2, 89-97 of L3, 31-35B of H1, 50-65 of H2, and 95-102 of H3. (Kabat et al., Sequences of Proteins of Immunological Interest, 5th Ed. Public Health Service, National Institutes of Health, Bethesda, Md. (1991)). The terms hypervariable regions (HVRs) and complementarity determining regions (CDRs) both refer to portions of the variable region that form the antigen binding regions.

A “chimeric antibody” as used herein refers to an antibody comprising at least one variable region from a first species (such as mouse, rat, cynomolgus monkey, etc.) and at least one constant region from a second species (such as human, cynomolgus monkey, etc.). In some embodiments, a chimeric antibody comprises at least one mouse variable region and at least one human constant region. In some embodiments, a chimeric antibody comprises at least one cynomolgus variable region and at least one human constant region. In some embodiments, a chimeric antibody comprises at least one rat variable region and at least one mouse constant region. In some embodiments, all of the variable regions of a chimeric antibody are from a first species and all of the constant regions of the chimeric antibody are from a second species.

A “humanized antibody” as used herein refers to an antibody in which at least one amino acid in a framework region of a non-human variable region has been replaced with the corresponding amino acid from a human variable region. In some embodiments, a humanized antibody comprises at least one human constant region or fragment thereof. In some embodiments, a humanized antibody is a Fab, an scFv, a (Fab′)₂, etc.

A “CDR-grafted antibody” or “HVR-grafted antibody” as used herein refers to a humanized antibody in which the complementarity determining regions (CDRs) or hypervariable regions (HVRs) of a first (non-human) species have been grafted onto the framework regions (FRs) of a second (human) species.

A “human antibody” as used herein refers to antibodies produced in humans, antibodies produced in non-human animals that comprise human immunoglobulin genes, such as XenoMouse®, and antibodies selected using in vitro methods, such as phage display, wherein the antibody repertoire is based on a human immunoglobulin sequences.

An “afucosylated” antibody or an antibody “lacking fucose” refers to an IgG1 or IgG3 isotype antibody that lacks fucose in its constant region glycosylation. Glycosylation of human IgG1 or IgG3 occurs at Asn297 (N297) as core fucosylated biantennary complex oligosaccharide glycosylation terminated with up to 2 Gal residues. In some embodiments, an afucosylated antibody lacks fucose at Asn297. These structures are designated as G₀, G1 (α1,6 or α1,3) or G2 glycan residues, depending on the amount of terminal Gal residues. See, e.g., Raju, T. S., BioProcess Int. 1: 44-53 (2003). CHO type glycosylation of antibody Fc is described, e.g., in Routier, F. H., Glycoconjugate J. 14: 201-207 (1997). Within a population of antibodies, the antibodies are considered to be afucosylated if <5% of the antibodies of the population comprise fucose at Asn297.

“Effector functions” refer to biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

“Antibody-dependent cell-mediated cytotoxicity” or “ADCC” refers to a form of cytotoxicity in which secreted Ig bound onto Fc receptors (FcRs) present on certain cytotoxic cells (e.g. NK cells, neutrophils, and macrophages) enable these cytotoxic effector cells to bind specifically to an antigen-bearing target cell and subsequently kill the target cell with cytotoxins. The primary cells for mediating ADCC, NK cells, express FcγRIII only, whereas monocytes express FcγRI, FcγRII, and FcγRIII FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 or 6,737,056 (Presta), may be performed. Useful effector cells for such assays include PBMC and NK cells. Alternatively, or additionally, ADCC activity of the molecule of interest may be assessed in vivo, e.g., in an animal model such as that disclosed in Clynes et al. Proc. Natl. Acad. Sci. (USA) 95:652-656 (1998). Additional antibodies with altered Fc region amino acid sequences and increased or decreased ADCC activity are described, e.g., in U.S. Pat. Nos. 7,923,538, and 7,994,290.

An antibody having an “enhanced ADCC activity” refers to an antibody that is more effective at mediating ADCC in vitro or in vivo compared to the parent antibody, wherein the antibody and the parent antibody differ in at least one structural aspect, and when the amounts of such antibody and parent antibody used in the assay are essentially the same. In some embodiments, the antibody and the parent antibody have the same amino acid sequence, but the antibody is afucosylated while the parent antibody is fucosylated. In some embodiments, ADCC activity will be determined using the in vitro ADCC assay such as disclosed in U.S. Publication No. 2015-0050273-A1, but other assays or methods for determining ADCC activity, e.g. in an animal model, are contemplated. In other embodiments, an antibody with enhanced ADCC activity also has enhanced affinity for Fc gamma RIIIA In some embodiments, an antibody with enhanced ADCC activity has enhanced affinity for Fc gamma RIIIA (V158). In certain embodiments, an antibody with enhanced ADCC activity has enhanced affinity for Fc gamma RIIIA (F158).

“Enhanced affinity for Fc gamma RIIIA” refers to an antibody that has greater affinity for Fc gamma RIIIA (also referred to, in some instances, as CD16a) than a parent antibody, wherein the antibody and the parent antibody differ in at least one structural aspect. In some embodiments, the antibody and the parent antibody have the same amino acid sequence, but the antibody is afucosylated while the parent antibody is fucosylated. Any suitable method for determining affinity for Fc gamma RIIIA may be used. In some embodiments, affinity for Fc gamma RIIIA is determined by a method described in U.S. Publication No. 2015-0050273-A1. In other embodiments, an antibody with enhanced affinity for Fc gamma RIIIA also has enhanced ADCC activity. In certain embodiments, an antibody with enhanced affinity for Fc gamma RIIIA has enhanced affinity for Fc gamma RIIIA (V158). In some embodiments, an antibody with enhanced affinity for Fc gamma RIIIA has enhanced affinity for Fc gamma RIIIA (F158).

The term “leader sequence” refers to a sequence of amino acid residues located at the N terminus of a polypeptide that facilitates secretion of a polypeptide from a mammalian cell. A leader sequence may be cleaved upon export of the polypeptide from the mammalian cell, forming a mature protein. Leader sequences may be natural or synthetic, and they may be heterologous or homologous to the protein to which they are attached. Nonlimiting exemplary leader sequences include leader sequences from heterologous proteins. In some embodiments, an antibody lacks a leader sequence. In other embodiments, an antibody comprises at least one leader sequence, which may be selected from native antibody leader sequences and heterologous leader sequences.

The term “isolated” as used herein refers to a molecule that has been separated from at least some of the components with which it is typically found in nature. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (for example, genomic DNA or mitochondrial DNA, in the case of a DNA polynucleotide) in which it is typically found in nature, or is separated from at least some of the components of the cell in which it was produced, e.g., in the case of an RNA polynucleotide. Thus, a DNA polynucleotide that is contained in a vector inside a host cell may be referred to as “isolated” so long as that polynucleotide is not found in that vector in nature.

The terms “subject” and “patient” are used interchangeably herein to refer to a human. In some embodiments, methods of treating other mammals, including, but not limited to, rodents, simians, felines, canines, equines, bovines, porcines, ovines, caprines, mammalian laboratory animals, mammalian farm animals, mammalian sport animals, and mammalian pets, are also provided.

The term “cancer” is used herein to refer to a group of cells that exhibit abnormally high levels of proliferation and growth. A cancer may be benign (also referred to as a benign tumor), pre-malignant, or malignant. Cancer cells may be solid cancer cells or leukemic cancer cells. The term “cancer growth” is used herein to refer to proliferation or growth by a cell or cells that comprise a cancer that leads to a corresponding increase in the size or extent of the cancer.

Examples of cancer include but are not limited to, carcinoma, lymphoma, blastoma, sarcoma, and leukemia. More particular nonlimiting examples of such cancers include squamous cell cancer, small-cell lung cancer, pituitary cancer, esophageal cancer, astrocytoma, soft tissue sarcoma, non-small cell lung cancer (including squamous cell non-small cell lung cancer), adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cell carcinoma, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, and various types of head and neck cancer (including squamous cell carcinoma of the head and neck).

“Treatment,” as used herein, refers to therapeutic treatment, for example, in order to cure a condition or disorder, to reduce in severity or slow progression of the condition or disorder, or to inhibit recurrence of the condition or disorder. In certain embodiments, the term “treatment” covers any administration or application of a therapeutic for disease in a patient, and includes inhibiting or slowing the disease or progression of the disease; partially or fully relieving the disease, for example, by causing regression, or restoring or repairing a lost, missing, or defective function; stimulating an inefficient process; or causing the disease plateau to have reduced severity. The term “treatment” also includes reducing the severity of any phenotypic characteristic and/or reducing the incidence, degree, or likelihood of that characteristic. Those in need of treatment include those already with the disorder as well as those at risk of recurrence of the disorder or those in whom a recurrence of the disorder is to be prevented or slowed down.

The term “effective amount” or “therapeutically effective amount” refers to an amount of a drug effective to treat a disease or disorder in a subject. In certain embodiments, an effective amount refers to an amount effective, at dosages and for periods of time necessary, to achieve the desired therapeutic result. A therapeutically effective amount of an anti-FGFR2 antibody of the invention may vary according to factors such as the disease state, age, sex, and weight of the individual, and the ability of the antibody or antibodies to elicit a desired response in the individual. A therapeutically effective amount encompasses an amount in which any toxic or detrimental effects of the antibody or antibodies are outweighed by the therapeutically beneficial effects. In some embodiments, the expression “effective amount” refers to an amount of the antibody that is effective for treating the cancer.

Additional definitions are provided in the sections that follow.

### Anti-FGFR2 Antibodies

In any of the formulations described herein involving an anti-FGFR2 antibody, the anti-FGFR2 antibody may be a monoclonal antibody, a genetically engineered antibody, a humanized antibody, a chimeric antibody, or a human antibody. In any of the compositions or methods described herein, the anti-FGFR2 antibody may be selected from a Fab, an Fv, an scFv, a Fab′, and a (Fab′) 2. In any of the compositions or formulations described herein, the anti-FGFR2 antibody may be selected from an IgA, an IgG, and an IgD. In any of the compositions or formulations described herein, the anti-FGFR2 antibody may be an IgG. In any of the methods described herein, the antibody may be an IgG1 or IgG3.

Exemplary anti-FGFR2 antibodies include antibodies that bind FGFR2-IIIb. In some embodiments, the anti-FGFR2-IIIb antibodies bind FGFR2-IIIc with lower affinity than they bind to FGFR2-IIIb. In other embodiments, the anti-FGFR2-IIIb antibodies do not detectably bind to FGFR2-IIIc.

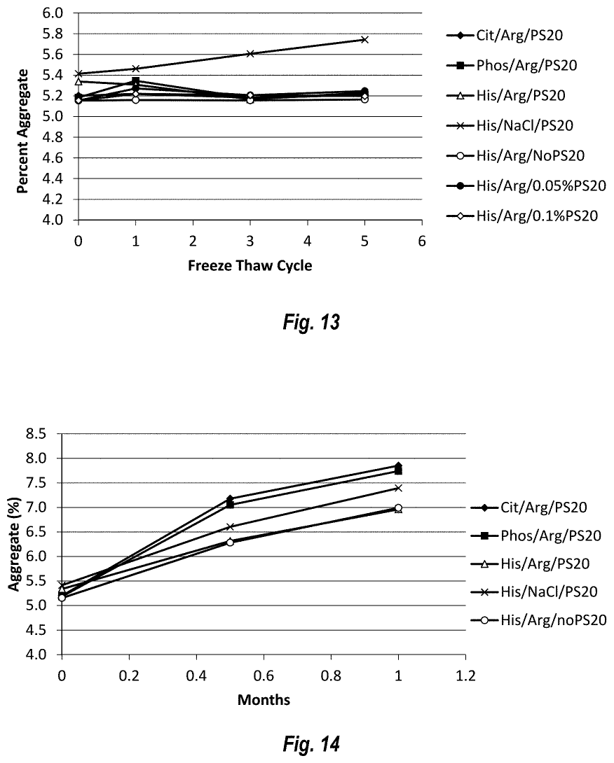

An exemplary anti-FGFR2-IIIb antibody for use in the embodiments herein is the HuGAL-FR21 antibody described in U.S. Pat. No. 8,101,723 B2, issued Jan. 24, 2012, which is specifically incorporated herein by reference. FIGS. 13 and 14 of U.S. Pat. No. 8,101,723 B2 show the amino acid sequences of the variable regions and full-length mature antibody chains of HuGAL-FR21, and are incorporated by reference herein. The heavy chain variable region sequences of antibody HuGAL-FR21, are underlined in FIG. 13 of U.S. Pat. No. 8,101,723 B2, and are specifically incorporated by reference herein. In some embodiments, the antibody is afucosylated. In some embodiments, the antibody is an IgG1 or IgG3 antibody that lacks fucose at Asn297. Additional antibodies that may be used in the embodiments herein include those described in U.S. Patent Publication No. 2015-0050273-A1, which describes certain afucosylated FGFR2-IIIb antibodies, and which is incorporated by reference herein.

In some embodiments, the anti-FGFR2-IIIb antibody comprises at least one, two, three, four, five, or six hypervariable regions (HVRs; e.g., CDRs) selected from (a) HVR-H1 comprising the amino acid sequence of SEQ ID NO: 6; (b) HVR-H2 comprising the amino acid sequence of SEQ ID NO: 7; (c) HVR-H3 comprising the amino acid sequence of SEQ ID NO: 8; (d) HVR-L1 comprising the amino acid sequence of SEQ ID NO: 9; (e) HVR-L2 comprising the amino acid sequence of SEQ ID NO: 10; and (f) HVR-L3 comprising the amino acid sequence of SEQ ID NO: 11. In some embodiments, the antibody is afucosylated. In some embodiments, the antibody is an IgG1 or IgG3 antibody that lacks fucose at Asn297.

In some embodiments, the anti-FGFR2-IIIb antibody comprises a heavy chain variable region and a light chain variable region. In some embodiments, the anti-FGFR2-IIIb antibody comprises at least one heavy chain comprising a heavy chain variable region and at least a portion of a heavy chain constant region, and at least one light chain comprising a light chain variable region and at least a portion of a light chain constant region. In some embodiments, the anti-FGFR2-IIIb antibody comprises two heavy chains, wherein each heavy chain comprises a heavy chain variable region and at least a portion of a heavy chain constant region, and two light chains, wherein each light chain comprises a light chain variable region and at least a portion of a light chain constant region. In some embodiments, the anti-FGFR2-IIIb antibody comprises a heavy chain variable region comprising the amino acid sequence of SEQ ID NO: 4 and a light chain variable region comprising the amino acid sequence of SEQ ID NO: 5. In some embodiments, the anti-FGFR2-IIIb antibody comprises a heavy chain comprising the amino acid sequence of SEQ ID NO: 2 and a light chain comprising the amino acid sequence of SEQ ID NO: 3. In some embodiments, the antibody is afucosylated. In some embodiments, the antibody is an IgG1 or IgG3 antibody that lacks fucose at Asn297.

In some embodiments, the anti-FGFR2-IIIb antibody comprises six HVRs comprising (a) HVR-H1 comprising the amino acid sequence of SEQ ID NO: 6; (b) HVR-H2 comprising the amino acid sequence of SEQ ID NO: 7; (c) HVR-H3 comprising the amino acid sequence of SEQ ID NO: 8; (d) HVR-L1 comprising the amino acid sequence of SEQ ID NO: 9; (e) HVR-L2 comprising the amino acid sequence of SEQ ID NO: 10; and (f) HVR-L3 comprising the amino acid sequence of SEQ ID NO: 11. In some embodiments, the anti-FGFR2-IIIb antibody comprises the six HVRs as described above and binds to FGFR2-IIIb. In some embodiments, the anti-FGFR-IIIb antibody does not bind to FGFR2-IIIc. In some embodiments, the antibody is afucosylated. In some embodiments, the antibody is an IgG1 or IgG3 antibody that lacks fucose at Asn297.

In one aspect, the anti-FGFR2-IIIb antibody competes with an anti-FGFR2-IIIb antibody comprising six HVRs comprising (a) HVR-H1 comprising the amino acid sequence of SEQ ID NO: 6; (b) HVR-H2 comprising the amino acid sequence of SEQ ID NO: 7; (c) HVR-H3 comprising the amino acid sequence of SEQ ID NO: 8; (d) HVR-L1 comprising the amino acid sequence of SEQ ID NO: 9; (e) HVR-L2 comprising the amino acid sequence of SEQ ID NO: 10; and (f) HVR-L3 comprising the amino acid sequence of SEQ ID NO: 11. In some embodiments, the antibody is afucosylated. In some embodiments, the antibody is an IgG1 or IgG3 antibody that lacks fucose at Asn297.

In some embodiments, the anti-FGFR2-IIIb antibody comprises at least one, at least two, or all three VH HVR sequences selected from (a) HVR-H1 comprising the amino acid sequence of SEQ ID NO: 6; (b) HVR-H2 comprising the amino acid sequence of SEQ ID NO: 7; and (c) HVR-H3 comprising the amino acid sequence of SEQ ID NO: 8. In some embodiments, the antibody is afucosylated. In some embodiments, the antibody is an IgG1 or IgG3 antibody that lacks fucose at Asn297.

In some embodiments, the anti-FGFR2-IIIb antibody comprising at least one, at least two, or all three VL HVR sequences selected from (a) HVR-L1 comprising the amino acid sequence of SEQ ID NO: 9; (b) HVR-L2 comprising the amino acid sequence of SEQ ID NO: 10; and (c) HVR-L3 comprising the amino acid sequence of SEQ ID NO: 11. In some embodiments, the antibody is afucosylated. In some embodiments, the antibody is an IgG1 or IgG3 antibody that lacks fucose at Asn297.

In some embodiments, the anti-FGFR2-IIIb antibody comprises (a) a VH domain comprising at least one, at least two, or all three VH HVR sequences selected from (i) HVR-H1 comprising the amino acid sequence of SEQ ID NO: 6, (ii) HVR-H2 comprising the amino acid sequence of SEQ ID NO: 7, and (iii) HVR-H3 comprising an amino acid sequence selected from SEQ ID NO: 8; and (b) a VL domain comprising at least one, at least two, or all three VL HVR sequences selected from (i) HVR-L1 comprising the amino acid sequence of SEQ ID NO: 9, (ii) HVR-L2 comprising the amino acid sequence of SEQ ID NO: 10, and (c) HVR-L3 comprising the amino acid sequence of SEQ ID NO: 11. In some embodiments, the antibody is afucosylated. In some embodiments, the antibody is an IgG1 or IgG3 antibody that lacks fucose at Asn297.

In some embodiments, the anti-FGFR2-IIIb antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 4. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-FGFR2-IIIb antibody comprising that sequence retains the ability to bind to FGFR2-IIIb. In certain embodiments, such FGFR2-IIIb antibody retains the ability to selectively bind to FGFR2-IIIb without detectably binding to FGFR2-IIIc. In certain embodiments, a total of 1 to 10 amino acids have been substituted, inserted and/or deleted in SEQ ID NO: 4. In certain embodiments, substitutions, insertions, or deletions occur in regions outside the HVRs (i.e., in the FRs). Optionally, the anti-FGFR2-IIIb antibody comprises the VH sequence in SEQ ID NO: 5, including post-translational modifications of that sequence. In a particular embodiment, the VH comprises one, two or three HVRs selected from: (a) HVR-H1 comprising the amino acid sequence of SEQ ID NO: 6; (b) HVR-H2 comprising the amino acid sequence of SEQ ID NO: 7; and (c) HVR-H3 comprising the amino acid sequence of SEQ ID NO: 8. In some embodiments, the antibody is afucosylated. In some embodiments, the antibody is an IgG1 or IgG3 antibody that lacks fucose at Asn297.

In some embodiments, the anti-FGFR2-IIIb antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 5. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-FGFR2-IIIb antibody comprising that sequence retains the ability to bind to FGFR2-IIIb. In certain embodiments, the anti-FGFR2-IIIb antibody retains the ability to selectively bind to FGFR2-IIIb without binding to FGFR2-IIIc. In certain embodiments, a total of 1 to 10 amino acids have been substituted, inserted and/or deleted in SEQ ID NO: 5. In certain embodiments, the substitutions, insertions, or deletions occur in regions outside the HVRs (i.e., in the FRs). Optionally, the anti-FGFR2-IIIb antibody comprises the VL sequence in SEQ ID NO: 4, including post-translational modifications of that sequence. In a particular embodiment, the VL comprises one, two or three HVRs selected from (a) HVR-L1 comprising the amino acid sequence of SEQ ID NO: 9; (b) HVR-L2 comprising the amino acid sequence of SEQ ID NO: 10; and (c) HVR-L3 comprising the amino acid sequence of SEQ ID NO: 11. In some embodiments, the antibody is afucosylated. In some embodiments, the antibody is an IgG1 or IgG3 antibody that lacks fucose at Asn297.

In some embodiments, the anti-FGFR2-IIIb antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 4 and a light chain variable domain (VL) having at least 90%, 91%, 92%, 93%, 94%, 95%, 96%, 97%, 98%, 99%, or 100% sequence identity to the amino acid sequence of SEQ ID NO: 5. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, and a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-FGFR2-IIIb antibody comprising that sequence retains the ability to bind to FGFR2-IIIb. In certain embodiments, such an anti-FGFR2-IIIb antibody retains the ability to selectively bind to FGFR2-IIIb without binding to FGFR2-IIIc. In certain embodiments, a total of 1 to 10 amino acids have been substituted, inserted and/or deleted in SEQ ID NO: 4. In certain embodiments, a total of 1 to 10 amino acids have been substituted, inserted and/or deleted in SEQ ID NO: 5. In certain embodiments, substitutions, insertions, or deletions occur in regions outside the HVRs (i.e., in the FRs). Optionally, the anti-FGFR2-IIIb antibody comprises the VH sequence in SEQ ID NO: 4 and the VL sequence of SEQ ID NO: 5, including post-translational modifications of one or both sequence. In a particular embodiment, the VH comprises one, two or three HVRs selected from: (a) HVR-H1 comprising the amino acid sequence of SEQ ID NO: 6; (b) HVR-H2 comprising the amino acid sequence of SEQ ID NO: 7; and (c) HVR-H3 comprising the amino acid sequence of SEQ ID NO: 8; and the VL comprises one, two or three HVRs selected from (a) HVR-L1 comprising the amino acid sequence of SEQ ID NO: 9; (b) HVR-L2 comprising the amino acid sequence of SEQ ID NO: 10; and (c) HVR-L3 comprising the amino acid sequence of SEQ ID NO: 11. In some embodiments, the antibody is afucosylated. In some embodiments, the antibody is an IgG1 or IgG3 antibody that lacks fucose at Asn297.

In some embodiments, the anti-FGFR2-IIIb antibody a VH as in any of the embodiments provided above, and a VL as in any of the embodiments provided above. In one embodiment, the antibody comprises the VH and VL sequences in SEQ ID NO: 4 and SEQ ID NO: 5, respectively, including post-translational modifications of those sequences. In some embodiments, the antibody is afucosylated. In some embodiments, the antibody is an IgG1 or IgG3 antibody that lacks fucose at Asn297.

In some embodiments, the anti-FGFR2-IIIb antibody comprises a heavy chain sequence having at least 90%, 91%, 92%, 93%, 94%, 95%, 96%, 97%, 98%, 99%, or 100% sequence identity to the amino acid sequence of SEQ ID NO: 2. In certain embodiments, a heavy chain sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-FGFR2-IIIb antibody comprising that sequence retains the ability to bind to FGFR2-IIIb. In certain embodiments, such an anti-FGFR2-IIIb antibody retains the ability to selectively bind to FGFR2-IIIb without detectably binding to FGFR2-IIIc. In certain embodiments, a total of 1 to 10 amino acids have been substituted, inserted and/or deleted in SEQ ID NO: 2. In certain embodiments, substitutions, insertions, or deletions occur in regions outside the HVRs (i.e., in the FRs). Optionally, the anti-FGFR2-IIIb antibody heavy chain comprises the VH sequence in SEQ ID NO: 2, including post-translational modifications of that sequence. In a particular embodiment, the heavy chain comprises one, two or three HVRs selected from: (a) HVR-H1 comprising the amino acid sequence of SEQ ID NO: 6; (b) HVR-H2 comprising the amino acid sequence of SEQ ID NO: 7; and (c) HVR-H3 comprising the amino acid sequence of SEQ ID NO: 8. In some embodiments, the antibody is afucosylated. In some embodiments, the antibody is an IgG1 or IgG3 antibody that lacks fucose at Asn297.

In some embodiments the anti-FGFR2-IIIb antibody comprises a light chain having at least 90%, 91%, 92%, 93%, 94%, 95%, 96%, 97%, 98%, 99%, or 100% sequence identity to the amino acid sequence of SEQ ID NO: 3. In certain embodiments, a light chain sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-FGFR2-IIIb antibody comprising that sequence retains the ability to bind to FGFR2-IIIb. In certain embodiments, such an anti-FGFR2-IIIb antibody retains the ability to selectively bind to FGFR2-IIIb without detectably binding to FGFR2-IIIc. In certain embodiments, a total of 1 to 10 amino acids have been substituted, inserted and/or deleted in SEQ ID NO: 3. In certain embodiments, the substitutions, insertions, or deletions occur in regions outside the HVRs (i.e., in the FRs). Optionally, the anti-FGFR2-IIIb antibody light chain comprises the VL sequence in SEQ ID NO: 3, including post-translational modifications of that sequence. In a particular embodiment, the light chain comprises one, two or three HVRs selected from (a) HVR-L1 comprising the amino acid sequence of SEQ ID NO: 9; (b) HVR-L2 comprising the amino acid sequence of SEQ ID NO: 10; and (c) HVR-L3 comprising the amino acid sequence of SEQ ID NO: 11. In some embodiments, the antibody is afucosylated. In some embodiments, the antibody is an IgG1 or IgG3 antibody that lacks fucose at Asn297.

In some embodiments, the anti-FGFR2-IIIb antibody comprises a heavy chain sequence having at least 90%, 91%, 92%, 93%, 94%, 95%, 96%, 97%, 98%, 99%, or 100% sequence identity to the amino acid sequence of SEQ ID NO: 2 and a light chain sequence having at least 90%, 91%, 92%, 93%, 94%, 95%, 96%, 97%, 98%, 99%, or 100% sequence identity to the amino acid sequence of SEQ ID NO: 3. In certain embodiments, a heavy chain sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-FGFR2-IIIb antibody comprising that sequence retains the ability to bind to FGFR2-IIIb. In certain embodiments, such an anti-FGFR2-IIIb antibody retains the ability to selectively bind to FGFR2-IIIb without detectably binding to FGFR2-IIIc. In certain embodiments, a light chain sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-FGFR2-IIIb antibody comprising that sequence retains the ability to bind to FGFR2-IIIb. In certain embodiments, such an anti-FGFR2-IIIb antibody retains the ability to selectively bind to FGFR2-IIIb without detectably binding to FGFR2-IIIc. In certain embodiments, a total of 1 to 10 amino acids have been substituted, inserted and/or deleted in SEQ ID NO: 2. In certain embodiments, a total of 1 to 10 amino acids have been substituted, inserted and/or deleted in SEQ ID NO: 3. In certain embodiments, substitutions, insertions, or deletions occur in regions outside the HVRs (i.e., in the FRs). Optionally, the anti-FGFR2-IIIb antibody heavy chain comprises the VH sequence in SEQ ID NO: 2, including post-translational modifications of that sequence and the anti-FGFR2-IIIb antibody light chain comprises the VL sequence in SEQ ID NO: 3, including post-translational modifications of that sequence. In a particular embodiment, the heavy chain comprises one, two or three HVRs selected from: (a) HVR-H1 comprising the amino acid sequence of SEQ ID NO: 6; (b) HVR-H2 comprising the amino acid sequence of SEQ ID NO: 7; and (c) HVR-H3 comprising the amino acid sequence of SEQ ID NO: 8; and the light chain comprises one, two or three HVRs selected from (a) HVR-L1 comprising the amino acid sequence of SEQ ID NO: 9; (b) HVR-L2 comprising the amino acid sequence of SEQ ID NO: 10; and (c) HVR-L3 comprising the amino acid sequence of SEQ ID NO: 11. In some embodiments, the antibody is afucosylated. In some embodiments, the antibody is an IgG1 or IgG3 antibody that lacks fucose at Asn297.

Additional exemplary anti-FGFR2 antibodies are the GAL-FR22 and GAL-FR23 antibodies described in U.S. Pat. No. 8,101,723 B2, incorporated by reference herein. The light and heavy chain variable regions of GAL-FR22, for example, are provided as SEQ ID NOs: 7 and 8 in U.S. Pat. No. 8,101,723 B2, while the Kabat CDRs and the light and heavy chain variable regions are also provided in FIG. 16 of that patent, which are incorporated by reference herein. The GAL-FR21, GAL-FR22 and GAL-FR23 producing hybridomas are deposited at the American Type Culture Collection, PO Box 1549, Manassas Va., USA, 20108, as ATCC Numbers 9586, 9587, and 9408, on Nov. 6, Nov. 6, and Aug. 12, 2008, respectively. Thus, in some embodiments, the anti-FGFR2 antibody is an antibody comprising the amino acid sequence of an antibody obtained from one of those three hybridoma strains.

The heavy and light chain variable regions of GAL-FR22 are also presented herein as SEQ ID NOs: 15 and 19, while the Kabat CDRs are presented herein as SEQ ID NOs: 16-19 and 20-22 herein. Thus, in some embodiments the anti-FGFR2-IIIb antibody heavy chain variable region comprises: (i) HVR1 (CDR1) comprising the amino acid sequence of SEQ ID NO: 16; (ii) HVR2 comprising the amino acid sequence of SEQ ID NO: 17; and (iii) HVR3 comprising the amino acid sequence of SEQ ID NO: 18; and the light chain variable region comprises: (iv) HVR1 comprising the amino acid sequence of SEQ ID NO: 20; (v) HVR2 comprising the amino acid sequence of SEQ ID NO: 21; and (vi) HVR3 comprising the amino acid sequence of SEQ ID NO: 22.

In some embodiments, the anti-FGFR2 antibody comprises an FGFR2-IIIb antibody in which the heavy chain variable domain that is at least 95%, such as at least 97%, at least 98%, or at least 99% identical to the amino acid sequence of SEQ ID NO:15, or that comprises the amino acid sequence of SEQ ID NO: 15. In some embodiments, the anti-FGFR2 antibody comprises an FGFR2-IIIb antibody in which the light chain variable domain is at least 95%, such as at least 97%, at least 98%, or at least 99% identical to the amino acid sequence of SEQ ID NO:19, or that comprises the amino acid sequence of SEQ ID NO: 19. In some embodiments, the heavy chain variable domain is at least 95%, such as at least 97%, at least 98%, or at least 99% identical to the amino acid sequence of SEQ ID NO:15, or that comprises the amino acid sequence of SEQ ID NO: 39 and the light chain variable domain is at least 95%, such as at least 97%, at least 98%, or at least 99% identical to the amino acid sequence of SEQ ID NO:19, or that comprises the amino acid sequence of SEQ ID NO: 19. In some such embodiments, the anti-FGFR2-IIIb antibody heavy chain variable region also comprises: (i) HVR1 (CDR1) comprising the amino acid sequence of SEQ ID NO: 16; (ii) HVR2 comprising the amino acid sequence of SEQ ID NO: 17; and (iii) HVR3 comprising the amino acid sequence of SEQ ID NO: 18; and/or the light chain variable region also comprises: (iv) HVR1 comprising the amino acid sequence of SEQ ID NO: 20; (v) HVR2 comprising the amino acid sequence of SEQ ID NO: 21; and (vi) HVR3 comprising the amino acid sequence of SEQ ID NO: 22. In some embodiments, the antibody is an IgG1 or IgG3 antibody that lacks fucose at Asn297.

### Afucosylated Anti-FGFR2 Antibodies

In some embodiments, the anti-FGFR2 antibodies described herein have a carbohydrate structure that lacks fucose attached (directly or indirectly) to an Fc region, i.e., the antibodies are afucosylated. In some embodiments, the afucosylated antibody is an IgG1 or IgG3 antibody that lacks fucose at Asn297.

Herein, antibodies are considered to be “afucosylated” when a plurality of such antibodies comprises at least 95% afucosylated antibodies. The amount of fucose may be determined by calculating the average amount of fucose within the sugar chain at Asn297 relative to the sum of all glycostructures attached to Asn 297 (e.g., complex, hybrid and high mannose structures). Nonlimiting exemplary methods of detecting fucose in an antibody include MALDI-TOF mass spectrometry (see, e.g., WO 2008/077546), HPLC measurement of released fluorescently labeled oligosaccharides (see, e.g., Schneider et al., “N-Glycan analysis of monoclonal antibodies and other glycoproteins using UHPLC with fluorescence detection,” Agilent Technologies, Inc. (2012); Lines, J. Pharm. Biomed. Analysis, 14: 601-608 (1996); Takahasi, J. Chrom., 720: 217-225 (1996)), capillary electrophoresis measurement of released fluorescently labeled oligosaccharides (see, e.g., Ma et al., Anal. Chem., 71: 5185-5192 (1999)), and HPLC with pulsed amperometric detection to measure monosaccharide composition (see, e.g., Hardy, et al., Analytical Biochem., 170: 54-62 (1988)). In some embodiments, in a composition of afucosylated antibodies herein, fucose is not detectable by one or more of these methods.

Asn297 refers to the asparagine residue located at about position 297 in the Fc region (EU numbering of Fc region residues); however, in a given antibody sequence, Asn297 may also be located about ±3 amino acids upstream or downstream of position 297, i.e., between positions 294 and 300, due to minor sequence variations in antibodies. In an FGFR2-IIIb antibody described herein, Asn297 is found in the sequence QYNST, and is in bold and underlined in the Table of Sequences shown below, SEQ ID NO: 2.

Fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “afucosylated” or “fucose-deficient” antibodies include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing afucosylated antibodies include Lec13 CHO cells deficient in protein fucosylation (Ripka et al. Arch. Biochem. Biophys. 249:533-545 (1986); US Patent Application No. US 2003/0157108 A1, Presta, L; and WO 2004/056312 A1, Adams et al., especially at Example 11), and knockout cell lines, such as cell lines lacking a functional alpha-1,6-fucosyltransferase gene, FUT8, e.g., knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Anti-FGFR2 antibodies herein may also have bisected oligosaccharides, e.g., in which a biantennary oligosaccharide attached to the Fc region of the antibody is bisected by GlcNAc. Such antibodies may have reduced fucosylation and/or improved ADCC function. Examples of such antibodies are described, e.g., in WO 2003/011878 (Jean-Mairet et al.); U.S. Pat. No. 6,602,684 (Umana et al.); and US 2005/0123546 (Umana et al.). In some embodiments, FGFR2 antibodies have at least one galactose residue in the oligosaccharide attached to the Fc region. Such antibodies may have improved CDC function. Such antibodies are described, e.g., in WO 1997/30087 (Patel et al.); WO 1998/58964 (Raju, S.); and WO 1999/22764 (Raju, S.).

In certain embodiments of the invention, an afucosylated FGFR2 antibody mediates ADCC in the presence of human effector cells more effectively than an antibody with the same amino acid sequence that comprises fucose. Generally, ADCC activity may be determined using the in vitro ADCC assay disclosed in U.S. Publication No. 2015-0050273 A1, but other assays or methods for determining ADCC activity, e.g. in an animal model etc., are contemplated.

In some embodiments, the FGFR2 antibody comprises the heavy and light chain sequences of SEQ ID NOs: 2 and 3. In other embodiments, the antibody comprising the heavy and light chain sequences of SEQ ID NOs: 2 and 3 is afucosylated.

### Pharmaceutical Formulations of Anti-FGFR2 Antibodies

The present disclosure provides pharmaceutical formulations of anti-FGFR2 antibodies, which are suitable for administration to a human. The anti-FGFR2 antibodies may be any such antibodies disclosed herein. The amount and type of compositions in the pharmaceutical formulations are selected to provide maximal stability for the antibody and thus, for maximal shelf-life.

The pharmaceutical formulations may comprise, for example, anti-FGFR2 antibody, a buffer chosen from histidine, citrate, and phosphate, sucrose, and a surfactant. In some embodiments, the formulations consist essentially of anti-FGFR2 antibody, a buffer chosen from histidine, citrate, and phosphate, sucrose, and surfactant. The pharmaceutical formulations may alternatively comprise, for example, anti-FGFR2 antibody, a buffer chosen from histidine, citrate, and phosphate, arginine, and a surfactant. In some embodiments, the formulations consist essentially of anti-FGFR2 antibody, a buffer chosen from histidine, citrate, and phosphate, arginine, and surfactant. The pharmaceutical formulations may comprise, for example, anti-FGFR2 antibody, histidine, sucrose, and a surfactant. In some embodiments, the formulations consist essentially of anti-FGFR2 antibody, histidine, sucrose, and surfactant. The pharmaceutical formulations may alternatively comprise, for example, anti-FGFR2 antibody, histidine, arginine, and a surfactant. In some embodiments, the formulations consist essentially of anti-FGFR2 antibody, histidine, arginine, and surfactant. In some embodiments, the formulations are suitable for intravenous administration to a patient, for example, by infusion or injection. In other embodiments, the formulations are suitable for subcutaneous administration to a patient, for example, by injection. In certain embodiments, the formulations are isotonic with bodily fluids in the area of administration, such as human blood plasma or lymphatic fluid.

In some embodiments, anti-FGFR2 antibodies herein have been found to be soluble in aqueous formulations up to 180 mg/mL In some embodiments the formulations comprise 10-180 mg/mL anti-FGFR2 antibody, or 10-30 mg/mL anti-FGFR2 antibody, or, for example, 10-25 mg/mL, 20-30 mg/mL, 15-25 mg/mL, 10-15 mg/mL, 15-20 mg/mL, 20-25 mg/mL, 18-22 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, 25 mg/mL, 26 mg/mL, 27 mg/mL, 28 mg/mL, 29 mg/mL, or 30 mg/mL of anti-FGFR2 antibody. In some embodiments, the formulations comprise 20 mg/mL of antibody.

In some embodiments, the formulations comprise histidine, citrate, or phosphate as buffer. In some such cases, the formulation buffer consists essentially of histidine. In some embodiments, the formulation comprises 5-40 mM of histidine citrate or phosphate, such as 10-40 mM, 10-30 mM, 15-25 mM, 10-20 mM, 20-30 mM, 15-20 mM, 20-25 mM, 25-30 mM, or 18-22 mM. In some embodiments, the formulation comprises 10 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, or 30 mM histidine, citrate, or phosphate or a range bounded by two of those concentrations. In certain embodiments, the formulation comprises 20 mM histidine, citrate, or phosphate.

In some embodiments, the formulations comprise histidine as buffer. In some such embodiments, the formulations do not comprise citrate or phosphate. In some such cases, the formulation buffer consists essentially of histidine. In some embodiments, the formulations comprise citrate as buffer. In some such embodiments, the formulations do not comprise histidine or phosphate. In some such cases, the formulation buffer consists essentially of citrate. In some embodiments, the formulations comprise phosphate as buffer. In some such embodiments, the formulations do not comprise citrate or histidine. In some such cases, the formulation buffer consists essentially of phosphate.

In some embodiments comprising or consisting essentially of histidine as buffer, the formulation comprises 5-40 mM of histidine, such as 10-40 mM, 10-30 mM, 15-25 mM, 10-20 mM, 20-30 mM, 15-20 mM, 20-25 mM, 25-30 mM, or 18-22 mM histidine. In some embodiments, the formulation comprises 10 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, or 30 mM histidine or a range bounded by two of those concentrations. In some embodiments, the formulation comprises 20 mM histidine.

In some embodiments, the surfactant is polysorbate, such as polysorbate 20 or polysorbate 80. In some such cases, the formulation comprises 0.001-0.1%, 0.002-0.1%, 0.01-0.1%, 0.005% to 0.05%, 0.005%, 0.006%, 0.007%, 0.008%, 0.009%, 0.01%, 0.02%, 0.03%, 0.04%, 0.05%, 0.06%, 0.07%, 0.08%, 0.09%, or 0.1% polysorbate 20 or 80. In some embodiments, the formulation comprises 0.005% to 0.05% polysorbate 20, such as 0.008% to 0.012% polysorbate 20. In other embodiments, the formulation comprises 0.002-0.1% polysorbate 20. In some embodiments, the formulation comprises 0.01% polysorbate 20.

Formulations Comprising Sucrose

In some embodiments, the formulation comprises or consists essentially of a mixture of anti-FGFR2 antibody, histidine, polysorbate (e.g. polysorbate 20), and sucrose. Concentrations of antibody, histidine, and polysorbate may, for example, be as provided above. In some embodiments, sucrose may be found in an amount that provides a formulation isotonic with bodily fluids. In some embodiments, the formulation comprises 200-300 mM sucrose, such as 200-250 mM, 250-300 mM, 200 mM, 210 mM, 220 mM, 230 mM, 240 mM, 250 mM, 260 mM, 270 mM, 280 mM, 290 mM, or 300 mM. In some embodiments, the formulation comprises 250-300 mM sucrose. In other embodiments, the formulation comprises 260-280 mM sucrose. In certain embodiments, the formulation comprises 250-270 mM sucrose. In some embodiments, the formulation comprises 270-300 mM sucrose. In other embodiments, the formulation comprises 250 mM, 260 mM, 270 mM, or 280 mM sucrose. In certain embodiments, the formulation comprises 270 mM sucrose. In some embodiments, a formulation comprising sucrose does not comprise arginine.

In some embodiments, the pH range of the pharmaceutical formulation is 5.0 to 7.0. In some embodiments, the sucrose-based pharmaceutical formulation has a pH of 5.0-6.5, such as 5.0-6.0, 5.5-6.0, 5.5-6.5, 5.8-6.2, 5.0, 5.1, 5.2, 5.3, 5.4, 5.5, 5.6, 5.7, 5.8, 5.9, 6.0, 6.1, 6.2, 6.3, 6.4, or 6.5. In some embodiments, the pH is 5.8-6.2. In some embodiments, the pH is 6.0.

Formulations Comprising Arginine

In some embodiments, the formulation comprises or consists essentially of histidine, arginine, polysorbate (e.g. polysorbate 20), and the antibody. In some such embodiments, the formulation does not comprise sucrose.

In some embodiments, the arginine is found at 100-200 mM, such as 100-150 mM, 150-200 mM, 130-170 mM, or 140-160 mM. In certain embodiments, the arginine is present in a concentration of 100 mM, 110 mM, 120 mM, 130 mM, 140 mM, 150 mM, 160 mM, 170 mM, 180 mM, 190 mM, or 200 mm. In other embodiments, the arginine is found at 140 mM, 150 mM, or 160 mM. In some embodiments the arginine is present at 150 mM.

In some embodiments, the pH range of the pharmaceutical formulation is 5.0 to 7.0. In some embodiments, the arginine-based pharmaceutical formulation has a pH of 5.0-6.5, such as 5.0-6.0, 5.5-6.5, 5.5-6.0, 5.5-5.9, 5.6-5.8, 5.0, 5.1, 5.2, 5.3, 5.4, 5.5, 5.6, 5.7, 5.8, 5.9, 6.0, 6.1, 6.2, 6.3, 6.4, or 6.5. In some embodiments, the pH is 5.5-5.9. In some embodiments, the pH is 5.6-5.8. In some embodiments, the pH is 5.7.

### Specific Formulation Embodiments

In other embodiments, the pharmaceutical formulation comprises 10-30 mg/mL anti-FGFR2 antibody, 130-170 mM arginine or 250-290 mM sucrose, 0.002-0.1% polysorbate 20, and 10-30 mM of one or more buffers selected from histidine, citrate, and phosphate, wherein the formulation has a pH of 5.0 to 7.0. In some such cases, the pharmaceutical formulation consists essentially of 10-30 mg/mL anti-FGFR2 antibody, 130-170 mM arginine or 250-290 mM sucrose, 0.002-0.1% polysorbate 20, and 10-30 mM of one or more buffers selected from histidine, citrate, and phosphate, wherein the formulation has a pH of 5.0 to 7.0.

In some embodiments, the pharmaceutical formulation comprises 10-30 mg/mL anti-FGFR2 antibody, 130-170 mM arginine or 250-290 mM sucrose, 0.005-0.05% polysorbate 20, and 10-30 mM of one or more buffers selected from histidine, citrate, and phosphate, wherein the formulation has a pH of 5.0 to 6.5. In some embodiments, the pharmaceutical formulation consists essentially of 10-30 mg/mL anti-FGFR2 antibody, 130-170 mM arginine or 250-290 mM sucrose, 0.005-0.05% polysorbate 20, and 10-30 mM of one or more buffers selected from histidine, citrate, and phosphate, wherein the formulation has a pH of 5.0 to 6.5. In some embodiments, the pharmaceutical formulation comprises 10-30 mg/mL anti-FGFR2 antibody, 130-170 mM arginine or 250-290 mM sucrose, 0.005-0.05% polysorbate 20, and 10-20 mM of one or more buffers selected from histidine, citrate, and phosphate, wherein the formulation has a pH of 5.0 to 6.5. In some embodiments, the pharmaceutical formulation consists essentially of 10-20 mg/mL anti-FGFR2 antibody, 130-170 mM arginine or 250-290 mM sucrose, 0.005-0.05% polysorbate 20, and 10-30 mM of one or more buffers selected from histidine, citrate, and phosphate, wherein the formulation has a pH of 5.0 to 6.5.

In some embodiments, the pharmaceutical formulation comprises 10-30 mg/mL anti-FGFR2 antibody, 140-160 mM arginine or 260-280 mM sucrose, 0.005-0.05% polysorbate 20, and 15-25 mM of one or more buffers selected from histidine, citrate, and phosphate, wherein the formulation has a pH of 5.5 to 6.5. In some embodiments, the pharmaceutical formulation consists essentially of 10-30 mg/mL anti-FGFR2 antibody, 140-160 mM arginine or 260-280 mM sucrose, 0.005-0.05% polysorbate 20, and 15-25 mM of one or more buffers selected from histidine, citrate, and phosphate, wherein the formulation has a pH of 5.5 to 6.5. In some embodiments, the pharmaceutical formulation comprises 10-20 mg/mL anti-FGFR2 antibody, 140-160 mM arginine or 260-280 mM sucrose, 0.005-0.05% polysorbate 20, and 15-25 mM of one or more buffers selected from histidine, citrate, and phosphate, wherein the formulation has a pH of 5.5 to 6.5. In some embodiments, the pharmaceutical formulation consists essentially of 10-20 mg/mL anti-FGFR2 antibody, 140-160 mM arginine or 260-280 mM sucrose, 0.005-0.05% polysorbate 20, and 15-25 mM of one or more buffers selected from histidine, citrate, and phosphate, wherein the formulation has a pH of 5.5 to 6.5.

In some embodiments, the pharmaceutical formulation comprises 10-30 mg/mL anti-FGFR2 antibody, 150 mM arginine or 270 mM sucrose, 0.005-0.05% polysorbate 20, and 15-25 mM of one or more buffers selected from histidine, citrate, and phosphate, wherein the formulation has a pH of 5.5 to 6.5. In some embodiments, the pharmaceutical formulation consists essentially of 10-30 mg/mL anti-FGFR2 antibody, 150 mM arginine or 270 mM sucrose, 0.005-0.05% polysorbate 20, and 15-25 mM of one or more buffers selected from histidine, citrate, and phosphate, wherein the formulation has a pH of 5.5 to 6.5. In some embodiments, the pharmaceutical formulation comprises 10-20 mg/mL anti-FGFR2 antibody, 150 mM arginine or 270 mM sucrose, 0.005-0.05% polysorbate 20, and 15-25 mM of one or more buffers selected from histidine, citrate, and phosphate, wherein the formulation has a pH of 5.5 to 6.5. In some embodiments, the pharmaceutical formulation consists essentially of 10-20 mg/mL anti-FGFR2 antibody, 150 mM arginine or 270 mM sucrose, 0.005-0.05% polysorbate 20, and 15-25 mM of one or more buffers selected from histidine, citrate, and phosphate, wherein the formulation has a pH of 5.5 to 6.5.

In some embodiments, the pharmaceutical formulation comprises 10-30 mg/mL anti-FGFR2 antibody, 150 mM arginine or 270 mM sucrose, 0.005-0.05% polysorbate 20, and 15-25 mM histidine, wherein the formulation has a pH of 5.5 to 6.5. In some embodiments, the pharmaceutical formulation consists essentially of 10-30 mg/mL anti-FGFR2 antibody, 150 mM arginine or 270 mM sucrose, 0.005-0.05% polysorbate 20, and 15-25 mM histidine, wherein the formulation has a pH of 5.5 to 6.5. In some embodiments, the pharmaceutical formulation comprises 10-20 mg/mL anti-FGFR2 antibody, 150 mM arginine or 270 mM sucrose, 0.005-0.05% polysorbate 20, and 15-25 mM histidine, wherein the formulation has a pH of 5.5 to 6.5. In some embodiments, the pharmaceutical formulation consists essentially of 10-20 mg/mL anti-FGFR2 antibody, 150 mM arginine or 270 mM sucrose, 0.005-0.05% polysorbate 20, and 15-25 mM histidine, wherein the formulation has a pH of 5.5 to 6.5.

In some embodiments, the pharmaceutical formulation comprises 10-30 mg/mL anti-FGFR2 antibody, 150 mM arginine or 270 mM sucrose, 0.008-0.012% polysorbate 20, and 10-30 mM histidine, wherein the formulation has a pH of 5.5 to 6.5. In some embodiments, the pharmaceutical formulation consists essentially of 10-30 mg/mL anti-FGFR2 antibody, 150 mM arginine or 270 mM sucrose, 0.008-0.012% polysorbate 20, and 10-30 mM histidine, wherein the formulation has a pH of 5.5 to 6.5. In some embodiments, the pharmaceutical formulation comprises 10-20 mg/mL anti-FGFR2 antibody, 150 mM arginine or 270 mM sucrose, 0.008-0.012% polysorbate 20, and 10-30 mM histidine, wherein the formulation has a pH of 5.5 to 6.5. In some embodiments, the pharmaceutical formulation consists essentially of 10-20 mg/mL anti-FGFR2 antibody, 150 mM arginine or 270 mM sucrose, 0.008-0.012% polysorbate 20, and 10-30 mM histidine, wherein the formulation has a pH of 5.5 to 6.5.

In some embodiments, the pharmaceutical formulation comprises 10-30 mg/mL anti-FGFR2 antibody, 150 mM arginine or 270 mM sucrose, 0.008-0.012% polysorbate 20, and 15-25 mM histidine, wherein the formulation has a pH of 5.5 to 6.5. In some embodiments, the pharmaceutical formulation consists essentially of 10-30 mg/mL anti-FGFR2 antibody, 150 mM arginine or 270 mM sucrose, 0.008-0.012% polysorbate 20, and 15-25 mM histidine, wherein the formulation has a pH of 5.5 to 6.5. In some embodiments, the pharmaceutical formulation comprises 10-20 mg/mL anti-FGFR2 antibody, 150 mM arginine or 270 mM sucrose, 0.008-0.012% polysorbate 20, and 15-25 mM histidine, wherein the formulation has a pH of 5.5 to 6.5. In some embodiments, the pharmaceutical formulation consists essentially of 10-20 mg/mL anti-FGFR2 antibody, 150 mM arginine or 270 mM sucrose, 0.008-0.012% polysorbate 20, and 15-25 mM histidine, wherein the formulation has a pH of 5.5 to 6.5.

In some embodiments, the pharmaceutical formulation comprises 10-30 mg/mL anti-FGFR2 antibody, 150 mM arginine or 270 mM sucrose, 0.01% polysorbate 20, and 20 mM histidine, wherein the formulation has a pH of 5.5 to 6.5. In some embodiments, the pharmaceutical formulation consists essentially of 10-30 mg/mL anti-FGFR2 antibody, 150 mM arginine or 270 mM sucrose, 0.01% polysorbate 20, and 20 mM histidine, wherein the formulation has a pH of 5.5 to 6.5. In some embodiments, the pharmaceutical formulation comprises 10-20 mg/mL anti-FGFR2 antibody, 150 mM arginine or 270 mM sucrose, 0.01% polysorbate 20, and 20 mM histidine, wherein the formulation has a pH of 5.5 to 6.5. In some embodiments, the pharmaceutical formulation consists essentially of 10-20 mg/mL anti-FGFR2 antibody, 150 mM arginine or 270 mM sucrose, 0.01% polysorbate 20, and 20 mM histidine, wherein the formulation has a pH of 5.5 to 6.5.

In some embodiments, the pharmaceutical formulation comprises 10-30 mg/mL anti-FGFR2 antibody, 150 mM arginine, 0.01% polysorbate 20, and 20 mM histidine, wherein the formulation has a pH of 5.6 to 5.8 or of 5.7. In some embodiments, the pharmaceutical formulation consists essentially of 10-30 mg/mL anti-FGFR2 antibody, 150 mM arginine, 0.01% polysorbate 20, and 20 mM histidine, wherein the formulation has a pH of 5.6 to 5.8 or of 5.7. In some embodiments, the pharmaceutical formulation comprises 10-20 mg/mL anti-FGFR2 antibody, 150 mM arginine, 0.01% polysorbate 20, and 20 mM histidine, wherein the formulation has a pH of 5.6 to 5.8 or of 5.7. In some embodiments, the pharmaceutical formulation consists essentially of 10-20 mg/mL anti-FGFR2 antibody, 150 mM arginine, 0.01% polysorbate 20, and 20 mM histidine, wherein the formulation has a pH of 5.6 to 5.8 or of 5.7.

In some embodiments, the pharmaceutical formulation comprises 10-30 mg/mL anti-FGFR2 antibody, 270 mM sucrose, 0.01% polysorbate 20, and 20 mM histidine, wherein the formulation has a pH of 5.9 to 6.1 or of 6.0. In some embodiments, the pharmaceutical formulation consists essentially of 10-30 mg/mL anti-FGFR2 antibody, 270 mM sucrose, 0.01% polysorbate 20, and 20 mM histidine, wherein the formulation has a pH of 5.9 to 6.1 or of 6.0. In some embodiments, the pharmaceutical formulation comprises 10-20 mg/mL anti-FGFR2 antibody, 270 mM sucrose, 0.01% polysorbate 20, and 20 mM histidine, wherein the formulation has a pH of 5.9 to 6.1 or of 6.0. In some embodiments, the pharmaceutical formulation consists essentially of 10-20 mg/mL anti-FGFR2 antibody, 270 mM sucrose, 0.01% polysorbate 20, and 20 mM histidine, wherein the formulation has a pH of 5.9 to 6.1 or of 6.0.

In some embodiments, the pharmaceutical formulation consists essentially of 10-30 mg/mL anti-FGFR2 antibody, 270 mM sucrose, 0.01% polysorbate 20, and 20 mM histidine, wherein the formulation has a pH of 5.0 to 6.0. In some embodiments, the pharmaceutical formulation consists essentially of 10-30 mg/mL anti-FGFR2 antibody, 270 mM sucrose, 0.01% polysorbate 20, and 20 mM histidine, wherein the formulation has a pH of 5.5 to 6.0.

In some embodiments, the formulation does not comprise certain types of excipients. For example, in some embodiments, the formulation does not comprise one or more of: sugar alcohols, proteins other than the anti-FGFR2 antibody, surfactants other than polysorbates, and amino acids other than arginine and/or histidine. In certain embodiments that comprise arginine, the formulation does not comprise one or more of: sugars, sugar alcohols, proteins other than the anti-FGFR2 antibody, surfactants other than polysorbates, and amino acids other than arginine and histidine. In some embodiments that comprise sucrose, the formulation does not comprise one or more of: sugars other than the sucrose, sugar alcohols, proteins other than the anti-FGFR2 antibody, surfactants other than polysorbates, and amino acids other than histidine. In some embodiments comprising arginine, the formulation does not comprise any of: sugars, sugar alcohols, proteins other than the anti-FGFR2 antibody, surfactants other than polysorbates, and amino acids other than arginine and histidine. In some embodiments comprising sucrose, the formulation does not comprise any of: sugars other than sucrose, sugar alcohols, proteins other than the anti-FGFR2 antibody, surfactants other than polysorbates, and amino acids other than histidine. In certain embodiments, the formulation comprises no other buffer ingredients other than histidine, citrate, and/or phosphate. In certain embodiments comprising histidine, citrate, or phosphate as a buffer, the formulation comprises no other buffer ingredients. As noted earlier in this application, the expression “does not comprise” in this context means that the excluded ingredients are not present beyond trace levels, for example, due to contamination or impurities found in other purposefully added ingredients.

Exemplary Properties of Formulations

In some embodiments, the formulation is stored as a liquid and is not lyophilized prior to administration to a patient. In some embodiments, the formulation is a ready-to-use, liquid formulation, and thus, may be administered directly to a patient. A liquid formulation provides an advantage of being ready-to-use and more easily administered to a patient than lyophilized formulations. In other embodiments, the formulation is diluted in saline, water, or is mixed with other substances to dilute the protein prior to administration. For example, a formulation designed for IV infusion may be diluted in saline or another appropriate buffer in an IV bag prior to administration. In some embodiments, a liquid formulation such as a ready-to-use liquid formulation is administered intravenously directly, without dilution.

In some embodiments, the formulation is administered intravenously, such as by intravenous (IV) infusion. The administration can be in a hospital, clinic, outpatient, or other medical office setting. IV administration allows for administration of protein therapeutics in relatively large volumes of liquid.

In other embodiments, for higher protein (antibody) concentrations (e.g., above 300 mg/mL), the pharmaceutical formulation herein is injectable or is administered subcutaneously, such as by injection, for instance using a subcutaneous administration device. In some embodiments, the formulation is contained in a vial. In other embodiments, the vial is part of or is attached to a subcutaneous administration device such as a syringe and needle. In some embodiments, the vial is a single-use vial. Vials herein may hold, for example, 0.5 mL, 1 mL, 1.5 mL, 2 mL, or 3 mL of the formulation. In some embodiments, a complete, single dosage of anti-FGFR2 antibody may be administered from one two, or three subcutaneous administrations. Thus, in some such embodiments, a complete, single dose of antibody may be contained within one, two, or three single-use vials. In certain embodiments, the vial comprises 1-2 mL of the formulation. Thus, in some embodiments, a complete, single dose for a patient may be contained within a single 1 mL, 1.5 mL, 2 mL, 2.5 mL, or 3 mL vial or within two or three 0.5 mL, 1 mL, 0.5 mL, or 2 mL vials. In some embodiments, a liquid formulation is stored in the dark, e.g. stored in a vial in the dark.

In some embodiments, the pharmaceutical formulation herein remains stable in solution and suitable for pharmaceutical use after, for example, 3 months or 6 months at 40° C. In other embodiments, the pharmaceutical formulation herein remains stable in solution and suitable for pharmaceutical use after, for example, for 6 months at 5° C. and/or 25° C. In certain embodiments, protein aggregation in the formulation may increase by no more than 3%, 4%, 5%, 6%, or 7% after 1 month, 2 months, or 3 months storage at 40° C. In yet other embodiments, protein aggregation in the formulation may increase by no more than 3% or 4% after 1 month of storage at 40° C. and/or no more than 7% after 3 months storage at 40° C. In some embodiments, protein aggregation in the formulation may increase by no more than 2% or 2.5% after 3 months of storage at 25° C. In some embodiments, protein aggregation in the formulation may increase by no more than 2% after 6 months of storage at 5° C.

In addition to aggregates, charged variants of a protein may form during storage as a result of chemical changes such as deamidation and oxidation. For example, in some embodiments, acidic variants of the anti-FGFR2 antibody do not increase or decrease by more than 20% after 3 months storage at 25° C. In some embodiments, there is no change in the percentage of charged variants after 6 months storage at 5° C. Appearance of charge variants may be detected, for example, using cation- or anion-exchange chromatography.

In certain embodiments, the pharmaceutical composition comprises no more than 40% of acidic variants of the anti-FGFR2 antibody, and no more than 20% of basic variants of the anti-FGFR2 antibody following storage for 1 month at 40° C. In some embodiments, a pharmaceutical composition comprises 1% to 40%, 15% to 40%, or 20% to 40%, 20% to 30%, or 30% to 40% acidic variants of the antibody and/or 5% to 30%, 5% to 20%, 10% to 20%, 20% to 30%, or 15% to 20% of basic variants of the antibody after 1 months at 40° C. In some embodiments, a pharmaceutical composition comprises an anti-FGFR2 antibody, wherein the composition comprises 15% to 35%, 20% to 30%, or 25% to 30% acidic variants of the antibody, and/or 15% to 40%, 15% to 30%, 15% to 25%, 15% to 20%, or 20% to 25% of basic variants of the antibody or antigen-binding fragment thereof after 3 months at 25° C. In some embodiments, a pharmaceutical composition comprises an anti-FGFR2 antibody, wherein the composition comprises 15% to 30%, 15% to 25%, 20% to 30%, or 20% to 25% acidic variants of the antibody, and/or 20% to 35%, 20% to 30%, or 25% to 30% of basic variants of the antibody or antigen-binding fragment thereof after 6 months at 5° C.

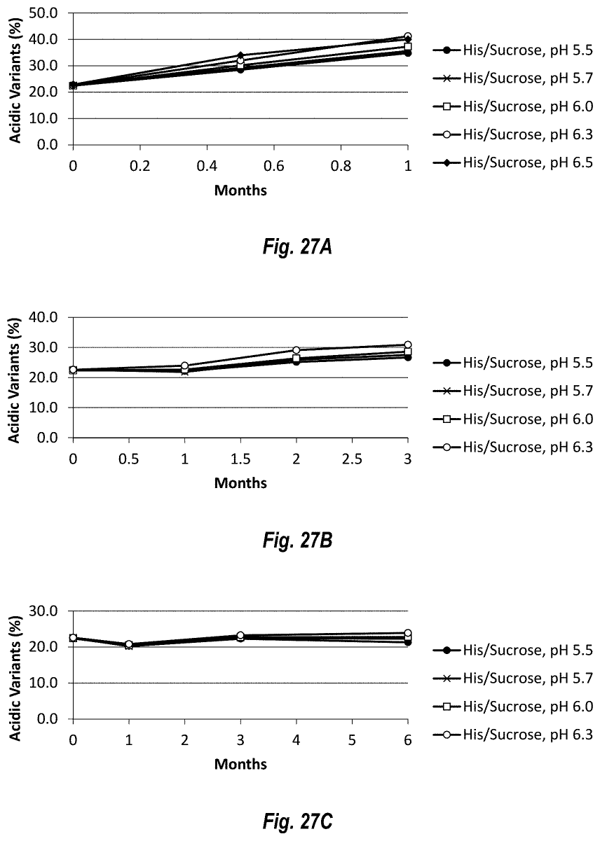

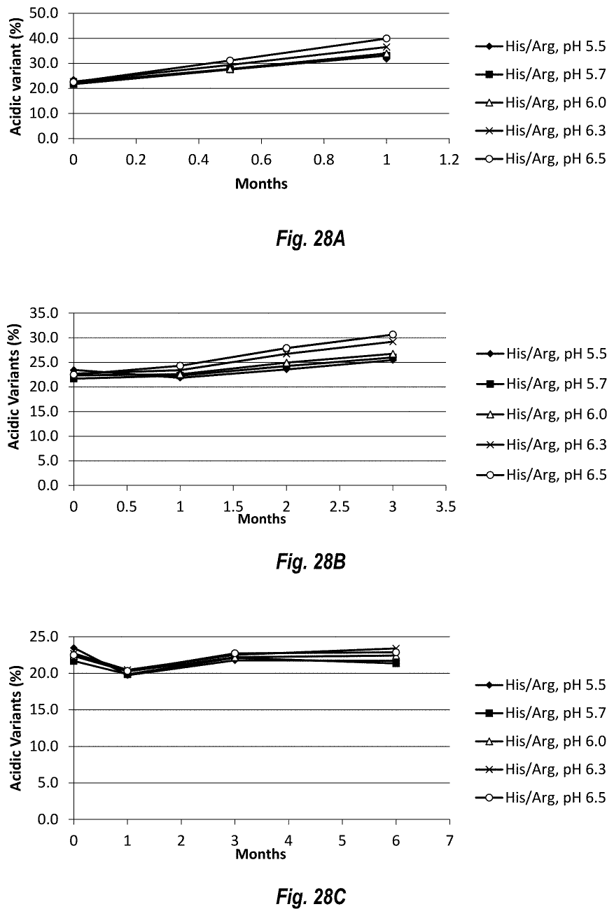

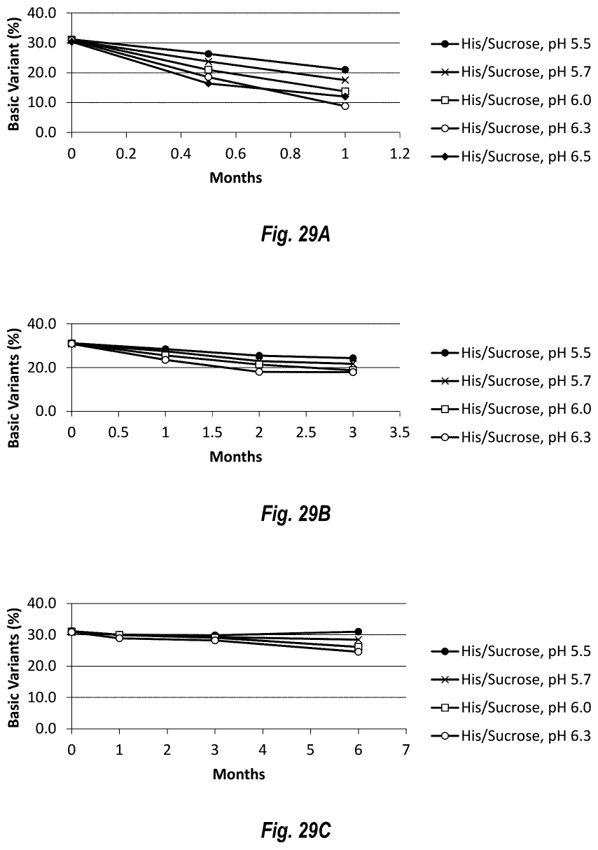

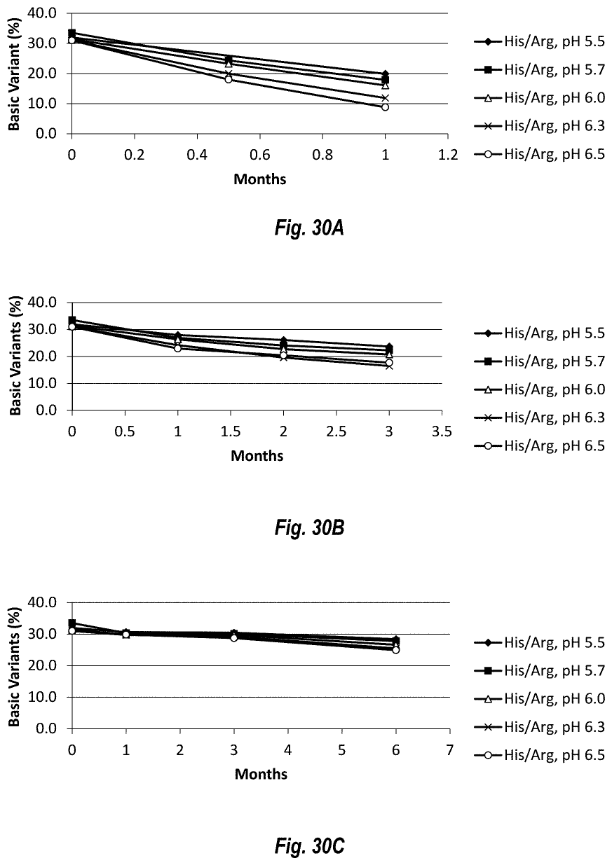

For example, in some embodiments, formulations comprising histidine, sucrose, polysorbate 20, at pH 5.5-6.5 herein initially contain 20-25% acidic variants of the anti-FGFR2 antibody while, following 1 month of storage at 40° C., contain between 20% and 40% acidic variants, after 3 months of storage at 25° C., contain between 25% and 30% acidic variants, and after 6 months of storage at 5° C., contain 20% to 25% acidic variants. (See FIG. 27A-C.) In some embodiments, formulations comprising histidine, arginine, polysorbate 20, at pH 5.5-6.5 herein initially contain 20-25% acidic variants of the anti-FGFR2 antibody while, following 1 month of storage at 40° C., contain between 20% and 40% acidic variants such as between 30% and 40%, after 3 months of storage at 25° C., contain between 25% and 30% acidic variants, and after 6 months of storage at 5° C., contain 20% to 25% acidic variants. (See FIG. 28A-C.) In some embodiments, formulations comprising histidine, sucrose, polysorbate 20, at pH 5.5-6.5 herein initially contain about 30% basic variants of the anti-FGFR2 antibody while, following 1 month of storage at 40° C., contain between 10% and 25% basic variants, after 3 months of storage at 25° C., contain between 15% and 30% such as 15% and 25% basic variants, and after 6 months of storage at 5° C., contain 25% to 30% basic variants. (See FIG. 29A-C.) In some embodiments, formulations comprising histidine, arginine, polysorbate 20, at pH 5.5-6.5 herein initially contain 30% to 35% basic variants of the anti-FGFR2 antibody while, following 1 month of storage at 40° C., contain between 10% and 20% basic variants, after 3 months of storage at 25° C., contain between 15% and 30% such as 15% and 25% basic variants, and after 6 months of storage at 5° C., contain 25% to 30% basic variants. (See FIG. 30A-C.)

In some embodiments, the pharmaceutical formulation remains stable for pharmaceutical use after one or more freeze-thaw cycles, such as after 2, 3, 4, or 5 freeze-thaw cycles, for example at −70° C. For example, in some embodiments, protein aggregation in the formulation may increase by no more than 2.0%, 1.5%, 1.0% or 0.5% after 5 freeze-thaw cycles. In certain embodiments, the percentage of high molecular weight protein species as measured by light scattering remains unchanged or increases by no more than 0.5% after 5 freeze-thaw cycles. In some embodiments, the formulation also remains stable to mechanical stress, which may be determined by exposing vials containing the formulation to agitation for an extended period of time. In other embodiments, aggregation may increase by no more than 2.0%, 1.5%, 1.0% or 0.5% after 72 hours of mechanical stress at 500 rpm.

### Methods of Treatment Using Anti-FGFR2 Antibody Pharmaceutical Formulations

Pharmaceutical formulations disclosed herein may be used in methods of treating patients in need of anti-FGFR2 antibody treatment. Uses of anti-FGFR2 antibodies are disclosed, for example, in International Patent Publication Nos. WO2015/017600 and WO2017/091577, which are each incorporated herein by reference.

In some embodiments, pharmaceutical formulations herein may be used to treat cancer patients. In some embodiments, the cancer is a solid tumor. More particular nonlimiting examples of such cancers include squamous cell cancer, small-cell lung cancer, pituitary cancer, esophageal cancer, astrocytoma, soft tissue sarcoma, non-small cell lung cancer (including squamous cell non-small cell lung cancer), adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cell carcinoma, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, and various types of head and neck cancer (including squamous cell carcinoma of the head and neck). In some embodiments, the cancer is gastric or bladder cancer.

In some embodiments, the cancer is recurrent or progressive after a therapy selected from surgery, chemotherapy, radiation therapy, or a combination thereof. In some embodiments, the subject is a PD-1/PD-L1 inhibitor inadequate responder. A subject who is a PD-1/PD-L1 inhibitor inadequate responder is one who may have previously responded to a PD-1/PD-L1 inhibitor, but may have become less responsive to the PD-1/PD-L1 inhibitor, or one who may have never responded to the PD-1/PD-L1 inhibitor. In some embodiments, the subject has previously received PD-1/PD-L1 inhibitor therapy.

In some embodiments, a cancer comprises an FGFR2 gene amplification. In some embodiments, a cancer comprising an FGFR2 gene amplification also overexpresses FGFR2IIIb. In some embodiments, a cancer comprising FGFR2 amplification overexpresses FGFR2IIIb to a greater extent than FGFR2IIIc. In some embodiments, a cancer comprising FGFR2 amplification expresses FGFR2IIIb at a normalized level that is more than 2-fold, 3-fold, 5-fold, or 10-fold greater than the normalized level of FGFR2IIIc expression. In some embodiments, the expression levels are normalized to GUSB. In some embodiments, a cancer overexpresses FGFR2IIIb but does not comprise FGFR2 gene amplification.

In some embodiments, the cancer is gastric cancer, which comprises an FGFR2 gene amplification. In some embodiments, a gastric cancer comprising an FGFR2 gene amplification overexpresses FGFR2IIIb. In some embodiments, a gastric cancer comprising FGFR2 amplification overexpresses FGFR2IIIb to a greater extent than FGFR2IIIc. In some embodiments, a gastric cancer comprising FGFR2 amplification expresses FGFR2IIIb at a normalized level that is more than 2-fold, 3-fold, 5-fold, or 10-fold greater than the normalized level of FGFR2IIIc expression. In some embodiments, the expression levels are normalized to GUSB. In some embodiments, a gastric cancer overexpresses FGFR2IIIb but does not comprise FGFR2 gene amplification. In some embodiments, overexpression is mRNA overexpression. In some embodiments, overexpression is protein overexpression.

FGFR2IIIb gene amplification may be determined by any suitable method in the art, including but not limited to, in situ hybridization (ISH). In some embodiments, FGFR2 amplification comprises FGFR2:CEN10 (chromosome 10 centromere) ratio of >3.

FGFR2IIIb mRNA overexpression may be determined by any suitable method in the art, including but not limited to, methods comprising quantitative PCR (qPCR). The term “FGFR2IIIb mRNA overexpression” means elevated levels of FGFR2IIIb mRNA, regardless of the cause of such elevated levels (i.e., whether the elevated levels are a result of increased transcription and/or decreased degradation of mRNA, other mechanism, or a combination of mechanisms).

FGFR2IIIb protein overexpression may be determined by any suitable method in the art, including but not limited to, antibody-based methods such as immunohistochemistry (IHC). In some embodiments, the IHC staining is scored according to methods in the art. The term “FGFR2IIIb protein overexpression” means elevated levels of FGFR2IIIb protein, regardless of the cause of such elevated levels (i.e., whether the elevated levels are a result of increased translation and/or decreased degradation of protein, other mechanism, or a combination of mechanisms). In some embodiments, 1+, 2+, or 3+ staining of tumor cells by IHC indicates FGFR2IIIb overexpression. For example, the overexpression may be determined by an IHC signal of 1+, 2+, or 3+ in at least 10% of tumor cells, such as in at least 20%, 30%, 40%, or 50% of tumor cells. In some embodiments, 2+ or 3+ staining of tumor cells by IHC indicates FGFR2IIIb overexpression. For example, the overexpression may be determined by an IHC signal of 2+ or 3+ in at least 10% of tumor cells, such as in at least 20%, 30%, 40%, or 50% of tumor cells.

In some embodiments, the FGFR2 overexpression may be reported as an “H score.” For example, in some such embodiments, the tumor is a bladder cancer tumor. To determine an H score, first membrane staining intensity may be determined for cells in a fixed field, such as via IHC to obtain scores of 0, 1+, 2+, or 3+ and the H score can be calculated using the formula as follows: 1×(% of cells visualized with IHC intensity of 1+)+2×(% of cells visualized with IHC intensity of 2+)+3×(% of cells visualized with IHC intensity of 3+). Theoretically, an H score may range from 0 to 300 and equals 300 if all of the cells in the visual field have IHC staining of 3+. In some embodiments, the patient to be treated has a starting H score for FGFR2, such as FGFR2b (e.g. FGFR2IIIb), of >20, such as >30, >40, >50, or >100, or a range of 20-300, 20-100, 20-50, 20-40, or 20-30. In some embodiments, the patient has an H score of >10 or is within a range of 10-20 or 15-20. In other embodiments, the patient has an H score of 0-10, which may indicate a lack of FGFR2 overexpression. In some such embodiments, the patient is a bladder cancer patient.

In some embodiments in which the patient suffers from gastric or bladder cancer, the subject may have been previously determined to have one of the following profiles or alternatively the method of treatment includes determining whether the patient fits one of the following profiles with respect to FGFR2 expression/gene amplification, and which may indicate the level of expected responsiveness to the treatment: a) in the case of a gastric cancer subject, an IHC signal of 3+ in at least 10% of tumor cells; b) in the case of a gastric cancer subject, an IHC signal of 3+ in at least 10% of tumor cells as well as amplification of the FGFR2 gene; c) in the case of a gastric cancer subject, an IHC signal of 3+ in at least 10% of tumor cells without amplification of the FGFR2 gene; d) in the case of a gastric cancer subject, an IHC signal of 1+ or 2+ in at least 10% of tumor cells; e) in the case of a bladder cancer subject, an IHC signal of 1+ in at least 10% of tumor cells; f) in the case of a bladder cancer subject, an IHC signal of 2+ in at least 10% of tumor cells; g) in the case of a bladder cancer subject, an H score of greater than 20; h) in the case of a bladder cancer subject, an H score of 10-19; i) in the case of a bladder cancer subject, an H score of less than 10. In some embodiments in which the patient suffers from gastric or bladder cancer, the subject may have been previously determined to have one of the following profiles or alternatively the method of treatment includes determining whether the patient fits one of the following profiles with respect to FGFR2 expression/gene amplification, and which may indicate the level of expected responsiveness to the treatment: a) in the case of a gastric cancer subject, an IHC signal of 3+ in at least 5% of tumor cells; b) in the case of a gastric cancer subject, an IHC signal of 3+ in at least 5% of tumor cells as well as amplification of the FGFR2 gene; c) in the case of a gastric cancer subject, an IHC signal of 3+ in at least 5% of tumor cells without amplification of the FGFR2 gene; d) in the case of a gastric cancer subject, an IHC signal of 1+ or 2+ in at least 5% of tumor cells; e) in the case of a bladder cancer subject, an IHC signal of 1+ in at least 5% of tumor cells; f) in the case of a bladder cancer subject, an IHC signal of 2+ in at least 5% of tumor cells; g) in the case of a bladder cancer subject, an H score of greater than 20; h) in the case of a bladder cancer subject, an H score of 10-19; i) in the case of a bladder cancer subject, an H score of less than 10. In some embodiments in which the patient suffers from gastric or bladder cancer, the subject may have been previously determined to have one of the following profiles or alternatively the method of treatment includes determining whether the patient fits one of the following profiles with respect to FGFR2 expression/gene amplification, and which may indicate the level of expected responsiveness to the treatment: a) the cancer is gastric cancer that has an FGFR2-IIIb immunohistochemistry (IHC) signal of 2+ or 3+ in a sample of the cancer; b) the cancer is gastric cancer that has an FGFR2-IIIb IHC signal of 2+ or 3+ in a sample of the cancer, and wherein the FGFR2 gene is amplified; c) the cancer is gastric cancer that has an FGFR2-IIIb IHC signal of 2+ or 3+ in a sample of the cancer, and wherein the FGFR2 gene is not amplified; or d) the cancer is bladder cancer that has an FGFR2-IIIb IHC signal of 2+ or 3+ in a sample of the cancer.

In some embodiments, the anti-FGFR2 antibody may be administered to a cancer patient at a dose of at least 0.1, 0.3, 0.5, 1, 2, 3, 4, 5, 10, 15, 20, 25, or 30 mg/kg, or within a range bounded by any two of those doses. In some embodiments, the antibody formulation is administered once per 1, 2, 3, 4, or 5 weeks.

In some embodiments, the pharmaceutical formulation comprising the anti-FGFR2 antibody is provided as part of a combination of agents, such as immune checkpoint inhibitors (immune stimulating agents) and chemotherapy agents. For example, anti-FGFR2 antibodies or formulations comprising them may be provided before, substantially contemporaneous with, or after other modes of treatment, for example, surgery, chemotherapy, or radiation therapy. In some embodiments, an effective amount of a formulation herein comprising an anti-FGFR2 antibody is administered in conjunction with another anti-cancer agent. Nonlimiting exemplary anti-cancer agents that may be administered with an anti-FGFR2 antibody include platinum agents (such as cisplatin, oxaliplatin, and carboplatin), paclitaxel (TAXOL®), albumin-engineered nanoparticle formulation of paclitaxel (ABRAXANE®), docetaxel (TAXOTERE®), gemcitabine (GEMZAR®), capecitabine (XELODA®), irinotecan (CAMPTOSAR®), epirubicin (ELLENCE®, PHARMORUBICIN®), FOLFOX (oxaliplatin combined with 5-FU and leucovorin), FOLFIRI (combination of leucovorin, 5-FU and irinotecan), leucovorin, fluorouracil (5-FU, EFUDEX®), mitomycin C (MITOZYTREX™, MUTAMYCIN®), and doxorubicin hydrochloride (Adriamycin PFS, Adriamycin RDF, RUBEX®). In some embodiments, an effective amount of a formulation comprising an anti-FGFR2 antibody is administered in conjunction with paclitaxel. In some embodiments, an effective amount of an anti-FGFR2 antibody formulation is administered in conjunction with cisplatin and/or 5-FU. In some embodiments, an effective amount of an anti-FGFR2 antibody formulation is administered in conjunction with FOLFOX (oxaliplatin, 5-FU, and leucovorin).

In some embodiments, methods for treating cancer are provided, comprising administering an effective amount of a pharmaceutical formulation comprising an anti-FGFR2 antibody and at least one immune stimulating agent. In some other embodiments, methods for treating cancer are provided, comprising administering an effective amount of a formulation comprising anti-FGFR2 antibody and an effective amount of at least one immune stimulating agent. In an exemplary embodiment, the at least one immune stimulating agent comprises a PD-1/PD-L1 inhibitor. In some embodiments, the formulation comprising the anti-FGFR2 antibody and the at least one immune stimulating agent, such as a PD-1/PD-L1 inhibitor, are administered concurrently. In some embodiments, the formulation comprising the anti-FGFR2 antibody and the at least one immune stimulating agent, such as a PD-1/PD-L1 inhibitor, are administered sequentially. In some embodiments, at least one, at least two, at least three doses, at least five doses, or at least ten doses of an anti-FGFR2 antibody is administered prior to administration of at least one immune stimulating agent, such as a PD-1/PD-L1 inhibitor. In some embodiments, at least one, at least two, at least three doses, at least five doses, or at least ten doses of at least one immune stimulating agent, such as a PD-1/PD-L1 inhibitor, is administered prior to administration of the formulation comprising the anti-FGFR2 antibody. In some embodiments, the last dose of at least one immune stimulating agent, such as a PD-1/PD-L1 inhibitor, is administered at least one, two, three, five, days or ten, or one, two, three, five, twelve, or twenty four weeks prior to the first dose of anti-FGFR2 antibody. In some other embodiments, the last dose of anti-FGFR2 antibody is administered at least one, two, three, five, days or ten, or one, two, three, five, twelve, or twenty four weeks prior to the first dose of at least one immune stimulating agent, such as a PD-1/PD-L1 inhibitor. In some embodiments, a subject has received, or is receiving, PD-1/PD-L1 inhibitor therapy, and an anti-FGFR2 antibody is added to the therapeutic regimen.

### EXAMPLES

The examples discussed below are intended to be purely exemplary of the invention and should not be considered to limit the invention in any way. Efforts have been made to ensure accuracy with respect to numbers used (for example, amounts, temperature, etc.) but some experimental errors and deviations should be accounted for.

### Example 1: Materials and Methods Used in Formulation Studies

I. Antibody Production

The anti-FGFR2-IIIb antibodies used in the following examples were produced in a Chinese hamster ovary (CHO) cell line that lacks the FUT8 gene (α1, 6-fucosyltransferase), and various lots that underwent changes in fermentation and purification processes were used in the development of the antibodies. The anti-FGFR2-IIIb antibody is an afucosylated, humanized IgG1 monoclonal antibody. A knowledge-based formulation development approach was used to identify the appropriate compositions that provide maximal stability for the protein. To do this, both intrinsic properties of the molecule and extrinsic formulation components that could affect the stability of the protein are considered. The studies were conducted to identify the appropriate components to produce a liquid formulation at 10-20 mg/mL, which can be diluted for IV infusion.

Raw materials: L-histidine (PN H3911), L-histidine.HCl (PN H5659) and sodium chloride (PN 59888) were obtained from Sigma Aldrich. L-arginine.HCl (PN 2067-06), citric acid (PN 0119), sodium citrate (PN 3627), and polysorbate 20 (PN 4116) were obtained from JT Baker (Thermo Fischer Scientific).

Container/Closure: 3 cc Type I West Pharmaceutical glass vials (PN 68000368) and 13 mm stopper (PN 19700116) from West Pharmaceutical Services, Inc. (PA), polypropylene tubes and glass HPLC vials were used to fill the formulations.

II. General Formulation Procedure

Formulations were prepared by dialysis of the drug substance into target formulation buffers using MWCO 20 kD dialysis membrane. Formulations were filtered in a laminar flow hood using 0.2 μm filter units and filled into appropriate container/closure systems for stability evaluation. Individual vials were pulled from designated storage conditions at pre-determined time points for analyses.

III. Analytical Methods

Stability samples were analyzed using early development assays described below.

Visual Inspection:

Visual assessment was made against a black/white background under fluorescence lighting.

Protein Concentration:

Protein concentration was determined by ultraviolet absorbance at 280 nm using theoretical absorption coefficients of 1.43 cm-1 [g/L]-1. Samples were diluted to within the linear range of absorbance with the appropriate formulation buffer and measured against Dulbecco's Phosphate Buffer Saline (DPBS) water. OD measurements were taken on a Beckman Coulter DU800 UV-Vis Spectrophotometer (Beckman Coulter, Inc., CA).

pH:

Buffer pH was determined using a calibrated Beckman Coulter pHi560 pH meter (Beckman Coulter, Inc., CA).

Osmolality:

Buffer osmolality was measured by vapor pressure using a Wescor VAPRO® system (Wescor, Inc., UT).

Differential Scanning calorimetry (DSC) Analysis:

The High Sensitivity Differential Scanning calorimetry (HSDSC) analyses were performed with the VP-DSC (MicroCal, Northampton, Mass.). Approximately 0.5 mg of each sample was loaded into the sample cell with the same amount of matching dialysis buffer into the reference cell. The samples were scanned from 20 to 90° C. or 100° C. at a rate of 1° C./min. The data were analyzed with the Origin 7.0 data analysis software (OriginLab, MA).

Ion Exchange Chromatography (IEX):

Cation exchange chromatography is a method of separating protein variants based on their net surface charge. Although the protein's isoelectric point (pI, which is the pH at which a protein has no net charge) is determined by its primary amino acid sequence, the net charge of the protein variants is based on the isoelectric charge of the protein and on the running buffer pH. For example, at a running buffer pH7, about one unit below the antibody's pI of 8.4, the protein will carry a net positive charge, and electrostatically bind to oppositely and weakly charged acidic carboxylate functional groups located on the Propac WCX-10 column resin with a substrate of ethylvinylbenzene-divinylbenzene and crosslinking. With a high performance liquid chromatography system equipped with a UV detection at 280 nm, the protein variants were separated and eluted from the column when an increasing salt gradient is applied, where variants with the weakest ionic interactions started to elute first and variants that have a stronger ionic interaction eluted later with a higher salt concentration. The isoforms were sequentially eluted into three regions as acidic, main and basic where the highest peak is the main isoform peak. Based on peaks areas, each region had a calculated relative peak percentage. The Weak Cation Exchange HPLC method was performed using a Dionex WCX-10 ProPac™ 4×250 mm column (Thermo Fischer Scientific). Samples were run on Agilent 1200 Series HPLC's and chromatograms were integrated using Chemstation software (Agilent Technologies, CA). Mobile Phase A was 10 mM HEPES at pH 7.0, and Mobile Phase B was 10 mM HEPES, 100 mM sodium chloride at pH 7.0. A gradient method was used from 40-90% B in 28 minutes and UV detected at 280 nm. The acidic, basic and main peak percentages were reported from the total area percent of each peak related absorbance.

Size-Exclusion Chromatography (SEC):

Size exclusion chromatography is a method of separating proteins based on their size. The method relies on a porous resin to separate different sized molecules. Large molecules elute earlier as these molecules are unable to access the pores of the resin and proceed through the column without hindrance. Smaller molecules can access the resin pores and have an increased path length through the column due to the tortuosity within the resin particles. Thus, smaller molecules elute after the larger molecules. Two methods were used for Size Exclusion Chromatography. The first method utilized a Tosoh G3000SWXL 7.8×300 mm column with 100 mM sodium phosphate, 700 mM arginine, pH 6.8 as the mobile phase at 0.5 mL/min for 30 minutes (Sigma Aldrich, Inc., MO). The second method utilized a Sepax Zenix SEC-300 7.8 X 200 mm column with 100 mM sodium phosphate, 400 mM sodium chloride pH 6.8 as the mobile phase at 1 mL/min for 12 minutes (Sepax Technologies, Inc., Delaware). Samples were run on Agilent 1100 & 1200 Series HPLC's and chromatograms integrated using Chemstation software with detection set at 280 nm. The percentages of aggregate, low molecular weight (LMW), and main peak were reported from the total area percent of each peak related absorbance.

Light Scattering:

A spectrophotometer was used to measure ultraviolet absorbance at 280 nm (UV A280). Concentration of the antibody samples was calculated using Beer's law, A=ε l c (ε=extinction coefficient; l=length of solution the light passes through (path length of cuvette) (cm), and c=concentration of solution). The theoretical extinction coefficient was used for the antibody concentration determination (e.g., the theoretical extinction coefficient for the anti-FGFR2 antibody is 1.43 (OD·ml)/(mg·cm)). The concentration in mg/mL for each sample preparation was calculated as follows: Concentration=(Absorbance at A280−Absorbance at A350)×(Dilution Factor)/(Extinction Coefficient×Path Length). The average and standard deviation for each sample were calculated.

### Example 2: Solubility in Water

The anti-FGFR2-IIIb antibody was concentrated to approximately 100 mg/mL and dialyzed into water. The sample was then further concentrated to 180 mg/mL. The anti-FGFR2-IIIb antibody was soluble in water at 180 mg/mL. The maximum solubility was not reached.

### Example 3: Initial Freeze/Thaw Stability of Anti-FGFR2-IIIb Antibody

A freeze/thaw study was performed on the anti-FGFR2-IIIb antibody. The antibody was concentrated to 11.6 mg/mL and formulated into 1×PBS at pH 7.4 with and without 0.01% polysorbate 20. The formulated solution was filled into vials and subjected to up to 3 cycles of freeze/thaw from −70° C. to ambient temperature. Sample vials were analyzed by visual inspection and SE-HPLC. The freeze/thaw results are shown in Table 1. A slight increase of aggregate was observed over the 3 freeze/thaw cycles. The results suggest that 1×PBS is acceptable for a formulation.

TABLE 1
Impact of Freeze/thaw Cycles on the Aggregation
of the Anti-FGFR2-IIIb Antibody.

Peak area (%)

| | | No | 0.01% |
|---|---|---|---|
| | | polysorbate | polysorbate |
| Sample | Peak | 20 | 20 |
| 4° C. | Pre- peak | 2.5 | 2.7 |
| control | Main peak | 95.5 | 96.2 |
| | LMW Peak | 1.9 | 1.0 |
| 1x | Pre- peak | 2.7 | 3.0 |
| Freeze/Thaw | Main peak | 96.1 | 96.2 |
| | LMW Peak | 1.2 | 0.8 |
| 2x | Pre- peak | 2.9 | 3.4 |
| Freeze/Thaw | Main peak | 95.1 | 94.7 |
| | LMW Peak | 1.9 | 1.8 |
| 3x | Pre- peak | 3.0 | 3.5 |
| Freeze/Thaw | Main peak | 95.1 | 95.1 |
| | LMW Peak | 1.9 | 1.3 |

### Example 4: Oxidation Studies of Anti-FGFR2-IIIb Antibody

The anti-FGFR2-IIIb antibody has a total of 10 methionine residues; among them, 4 methionines are in each of the heavy chains, and none of the methionines are located in CDR1. In addition, there is one methionine in the light chain.

An experiment was performed to evaluate the susceptibility of the methionines to oxidation by incubating anti-FGFR2-IIIb antibody with 0.01%, 0.1%, or 1.0% hydrogen peroxide overnight at room temperature. The oxidized sampled were subsequently characterized by tryptic mapping LC/MS for qualitatively determination the oxidation level of each methionine residue. The activity of the oxidized samples was determined by cell-based assay for potency, and binding affinity analyzed by Biacore™ (GE Healthcare). The peptide map LC/MS characterization shown in Table 2 revealed that light chain methionine 5 and heavy chain methionine 81 were less susceptible to oxidation. Heavy chain methionine 249, 355 and 425 were more susceptible to oxidation. All of the oxidized samples showed comparable potency to the control by ELISA assay.

TABLE 2
Relative susceptibility of the anti-FGFR2-IIIb antibody
methionine residues to peroxide induced oxidation
determined by tryptic map LC/MS (In table, L =
light chain, M = Methionine, H = heavy chain).

| | | | | | | | Potency |
|---|---|---|---|---|---|---|---|
| | | | H | H | H | H | By |
| Sample | Condition | L M 5 | M81 | M249 | M355 | M425 | ELISA |
| 1 | Control | 0 | 0 | 13 | 0 | 0 | 84 |
| 2 | +0.01% | 0 | 0 | 100 | 26 | 60 | 102 |
| | H₂O₂ | | | | | | |
| 3 | +0.1% | 0 | 7 | 100 | 65 | 100 | 89 |
| | H₂O₂ | | | | | | |
| 4 | +1.0% | 0 | 18 | 100 | 100 | 100 | 83 |
| | H₂O₂ | | | | | | |

The binding of anti-FGFR2-IIIb antibody to FGFR2b and protein A was measured by Biacore. Oxidation of the heavy chain methionines did not interfere with the binding of anti-FGFR2-IIIb antibody to FGFR2b (FIG. 1 and Table 3). Increase in oxidation interfered with binding to protein A. Oxidation degradation of anti-FGFR2-IIIb antibody was not critical given the activity of the anti-FGFR2-IIIb antibody was not significantly affected in highly oxidized samples.

TABLE 3
Potency by Blocking ELISA for Force Oxidized Samples

| | Sample | IC50 (ng/mL) | | % Potency | |
|---|---|---|---|---|---|
| Condition | Number | Test | Reference | Assay 1 | Assay 2 |
| Control | Sample 1 | 27.2 | 21.5 | 79 | 89 |
| 0.01% H2O2 | Sample 2 | 20.8 | 20.4 | 98 | 106 |
| 0.1% H2O2 | Sample 3 | 25.9 | 20.2 | 78 | 100 |
| 1.0% H2O2 | Sample 4 | 25.7 | 18.7 | 73 | 93 |

### Example 5: Effect of Buffer pH on Protein Stability

Formulation pH affects protein stability. The pH of a formulation can influence biochemical degradation pathways such as deamidation, isomerization, and oxidation, as well as biophysical degradations such as aggregation and fragmentation due to interactions between proteins and with their environment. Buffer pH was studied as a formulation variable that can affect product stability. The impact of pH on anti-FGFR2-IIIb antibody conformational stability and chemical stability upon storage was evaluated. The anti-FGFR2-IIIb antibody was two-step purified and formulated into 9 different isotonic formulations (Formulations 1-9 in Table 4) with the pH value ranging from 4.0 to 8.0. The protein concentration of the buffer exchanged solutions was adjusted to 1 mg/mL, the solutions were filled into vials for DSC analyses, and isothermal stability assessment at 40° C. and 25° C. The formulations evaluated are listed in the Table 4.

TABLE 4
Formulations evaluated for effect of pH on protein stability

| | | | Conc. |
|---|---|---|---|
| ID | Formulation | pH | (mg/ml) |
| 1 | 20 mM NaCitrate/citric acid, 150 mM NaCl | 4.0 | 1.0 |
| 2 | 20 mM NaCitrate/citric acid, 150 mM NaCl | 5.0 | 1.0 |
| 3 | 20 mM NaCitrate/citric acid, 150 mM NaCl | 6.0 | 1.0 |
| 4 | 20 mM L-histidine, 150 mM L-arginine | 6.0 | 1.0 |
| 5 | 20 mM L-histidine, 150 mM NaCl | 6.0 | 1.0 |
| 6 | 20 mM L-histidine, 150 mM NaCl | 7.0 | 1.0 |
| 7 | 20 mM NaPhosphate, 150 mM NaCl | 6.0 | 1.0 |
| 8 | 20 mM NaPhosphate, 150 mM NaCl | 7.0 | 1.0 |
| 9 | 20 mM NaPhosphate, 150 mM NaCl | 8.0 | 1.0 |

Conformational Stability by Differential Scanning Calorimetry (DSC) Analysis

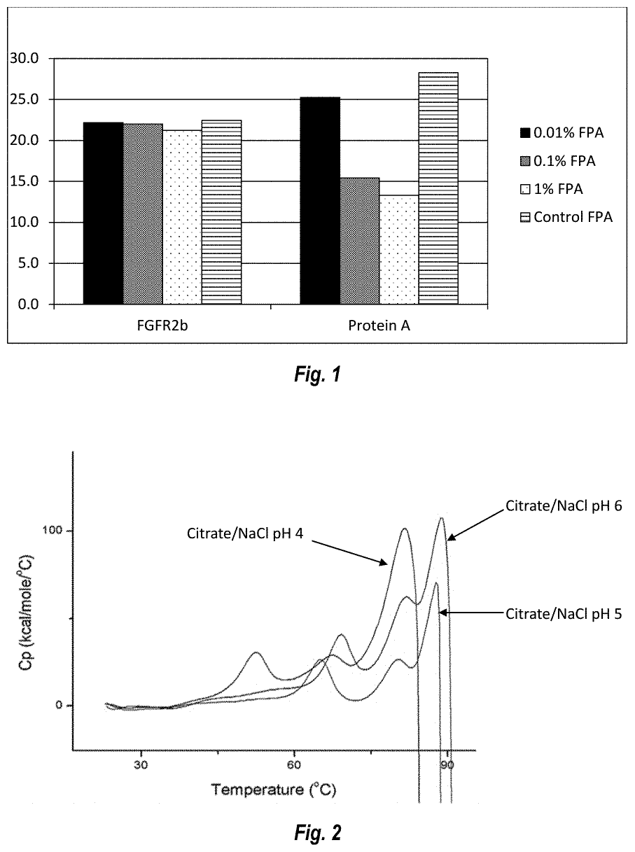

The DSC thermograms of formulations 1-9 from Table 4 were generated and analyzed. Formulations 1-3 are marked with a * in Table 5, and the results are shown in FIG. 2. Upon heating of the samples, three transitions were observed. After the unfolding transition, the IgG molecules formed insoluble aggregates and precipitated out of the solution. Table 5 shows that the unfolding temperature (T_{M}1) increased with the increase of pH and reached a maximum T_{M}1 at around 69° C. at pH 6 and pH 7. The buffer species did not show significant impact on the unfolding temperature.

TABLE 5
Unfolding temperature of anti-FGFR2-IIIb
antibody samples from pH screening study

| | | | | T_{M}1 | T_{M}2 | T_{M}3 |
|---|---|---|---|---|---|---|
| Formulation | Buffer | Bulking agent | pH | (° C.) | (° C.) | (° C.) |
| 1* | 20 mM Citrate | 150 mM NaCl | pH 4 | 52.7 | 68.5 | 81.0 |
| 2* | 20 mM Citrate | 150 mM NaCl | pH 5 | 64.7 | 80.3 | 87.5 |
| 3* | 20 mM Citrate | 150 mM NaCl | pH 6 | 70.0 | 81.7 | 89.2 |
| 4 | 20 mM Histidine | 150 mM Arg | pH 6 | 66.8 | 80.5 | 88.2 |
| 5 | 20 mM Histidine | 150 mM NaCl | pH 6 | 67.6 | 81.9 | 87.4 |
| 6 | 20 mM Histidine | 150 mM NaCl | pH 7 | 70.8 | 82.8 | 87.8 |
| 7 | 20 mM Phosphate | 150 mM NaCl | pH 6 | 70.5 | 82.5 | 87.9 |
| 8 | 20 mM Phosphate | 150 mM NaCl | pH 7 | 70.6 | 81.5 | 88.3 |
| 9 | 20 mM Phosphate | 150 mM NaCl | pH 8 | 70.3 | 81.0 | 88.0 |

Isothermal Stability

The impact of buffer pH on the formation of aggregates, clips (fragments), and charge variants were assessed by monitoring the sample stability under accelerated storage condition at 40° C. and 25° C.

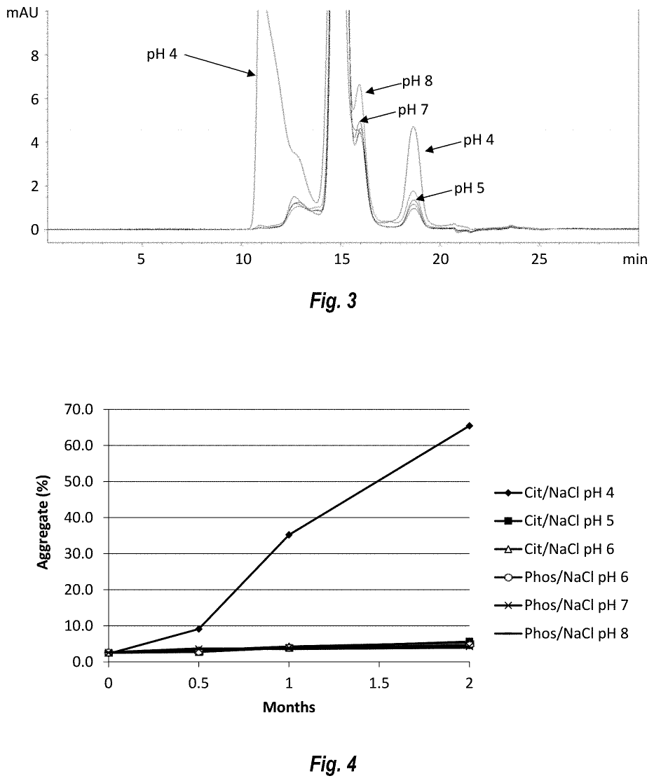

Changes in aggregates and clips were determined by SE-HPLC. Representative SE-HPLC chromatograms of selected anti-FGFR2-IIIb antibody pH screening samples are shown in FIG. 3. The main peak represents the monomer, the peaks eluted earlier than the monomer are aggregates, and the peaks eluted later are clips. The soluble aggregates increased rapidly in the pH 4 formulation at 40° C.; moderate increase of soluble aggregates with increasing pH was seen in pH 5.0 and pH 8.0 buffers. Similar effect of pH on the formation of clips was observed.

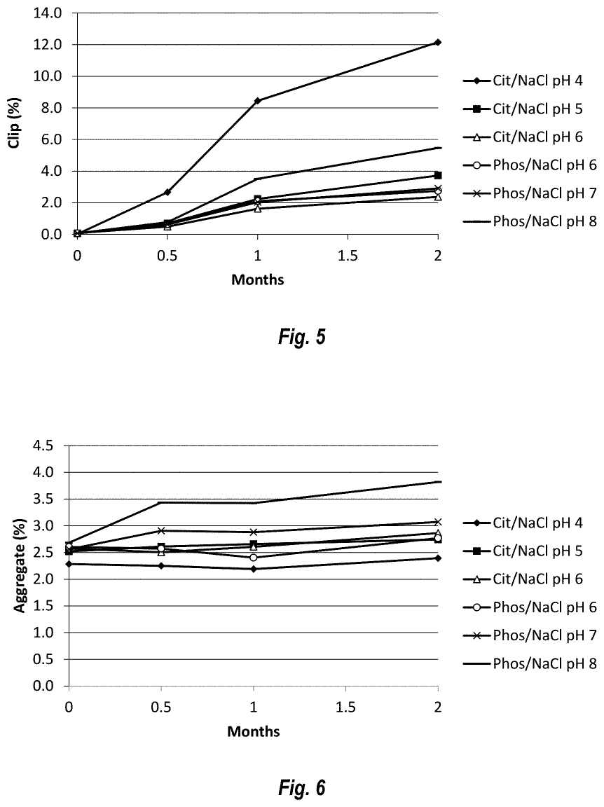

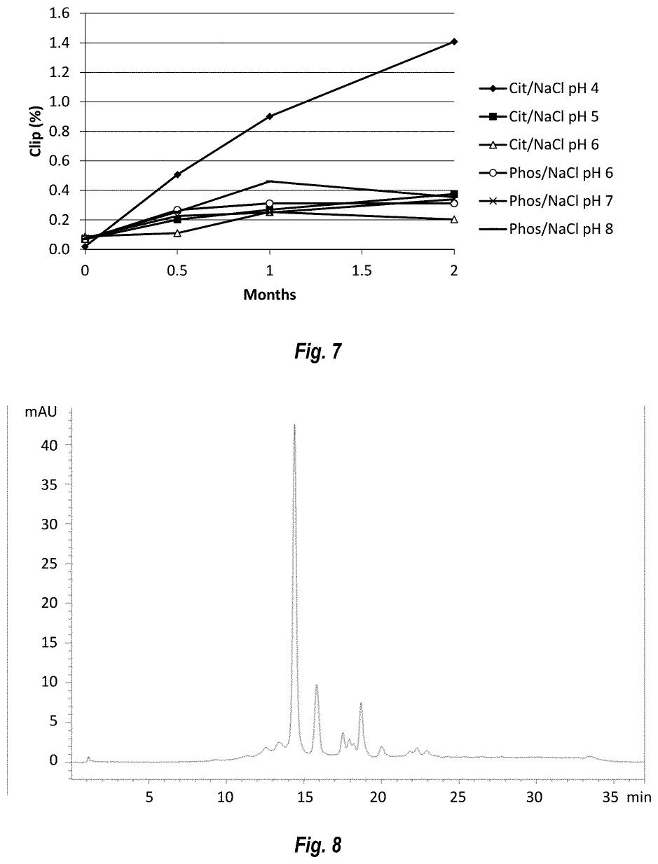

The impact of buffer pH on the aggregates (FIG. 4) and clips (FIG. 5) at 40° C. was analyzed by SE-HPLC. Formulations 1, 2, 3, 7, 8, and 9 listed in Table 5 were tested. Within the pH range tested, the clips formation was most significant at pH 4. At neutral pH, formation of clips was negligible even after storage at 40° C. for 1 month as shown in FIG. 5. No apparent increase in aggregates was observed in pH 5 to pH 8 formulations after at least 2 months storage at 25° C. (FIG. 6). There was no apparent increase in clips observed in the formulations at pH 5 to pH 8 after at least 2 months storage at 25° C. (FIG. 7).

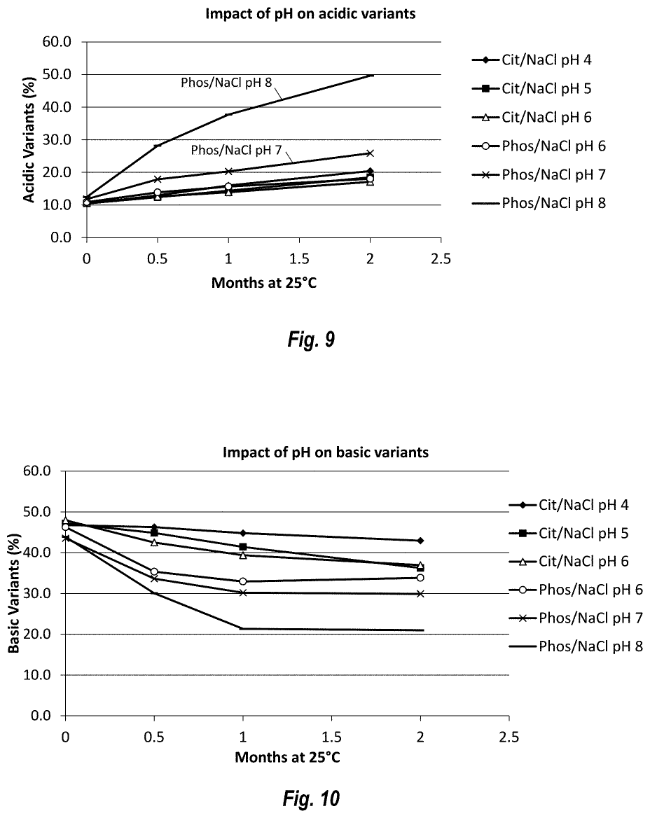

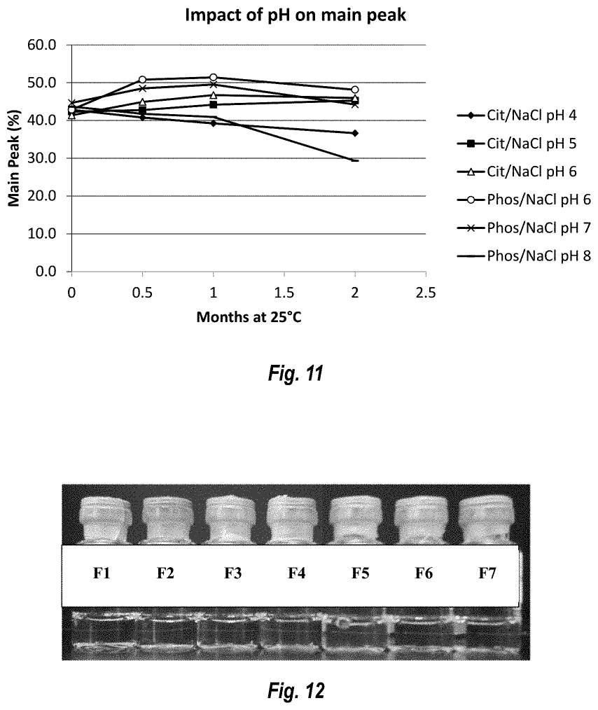

Change of charge variants distribution is another common degradation pathway for antibodies and is typically related to the formulation buffer pH. The charge variants of the anti-FGFR2-IIIb antibody were analyzed with a weak cation-exchange HPLC (WCX-HPLC) method. A representative chromatogram is shown in FIG. 8. The peaks appearing before the main peak are acidic species, and the peaks appearing after the main peak are basic species. The increase in acidic species is due to the deamidation of asparagines in the IgG molecule. The change of charge variants profile occurred drastically at 40° C. Accordingly, the change of charge profile was monitored at a lower temperature, such as 25° C. FIGS. 9-11 show the impact of buffer pH on the charge variants at 25° C. as determined by weak cation exchange high performance liquid chromatography (WCX-HPLC). As shown in FIG. 9, the acidic species increased rapidly in basic pH conditions, especially at pH 8.0. Samples formulated between pH 5 and pH 6 remained stable even after two months storage at 25° C. FIG. 10 shows the impact of pH on the percentage of basic variants from 0 to 2.5 months storage at 25° C. FIG. 11 shows the impact of pH on the main peak from 0 to 2.5 months storage at 25° C.

The results from the pH screening studies indicate that under stressed storage conditions, the stability of the anti-FGFR2-IIIb antibody was strongly dependent on the formulation pH. At basic pH, aggregation and formation of acidic variants were the major degradation pathway. The anti-FGFR2-IIIb antibody was determined to be most stable in the pH range of 5.0-6.0.

### Example 6: Effect of Excipients on Protein Stability

Formulation excipients, such as buffer species and bulking agents, were analyzed for their effects on product stability. To assess the effect of excipient on the stability of the anti-FGFR2-IIIb antibody, various citrate, phosphate, and histidine buffers were tested. Seven different isotonic solutions buffered at pH 6.0 that were tested are listed in Table 6.

TABLE 6
Formulations evaluated for effect of excipients on protein stability

| Formulation | | | Conc. |
|---|---|---|---|
| Number | Formulation | pH | (mg/ml) |
| 1 | 20 mM Citrate, 150 mM Arginine, 0.01% | 6.0 | 20 |
| | PS20 | | |
| 2 | 20 mM Phosphate, 150 mM Arginine, | 6.0 | 20 |
| | 0.01% PS20 | | |
| 3 | 20 mM Histidine, 150 mM Arginine, | 6.0 | 20 |
| | 0.01% PS20 | | |
| 4 | 20 mM Histidine, 150 mM Sodium | 6.0 | 20 |
| | Chloride, 0.01% PS20 | | |
| 5 | 20 mM Histidine, 150 mM Arginine, No | 6.0 | 20 |
| | PS20 | | |
| 6 | 20 mM Histidine, 150 mM Arginine, | 6.0 | 20 |
| | 0.05% PS20 | | |
| 7 | 20 mM Histidine, 150 mM Arginine, | 6.0 | 20 |
| | 0.10% PS20 | | |

The protein concentrations were adjusted to 20 mg/mL, and the solutions were filled into vials. Formulations 1˜4 in Table 6 were analyzed by DSC. The physical stability was evaluated for all formulations, and isothermal stability assessment at 40° C., 25° C. and 5° C. was determined for formulations 1-5.

Conformational Stability by DSC Analysis

The unfolding temperatures of various formulations of the anti-FGFR2-IIIb antibody were determined by DSC analysis and are summarized in Table 7. Precipitation was observed in all formulations after heating to 90° C. Surfactant in the formulation did not protect the antibody from precipitation after denaturing. As shown in Table 7, the formulations had comparable melting temperatures.

TABLE 7
Unfolding temperature of samples from excipient screening determined by DSC

| | | | | | T_{M}1 | T_{M}2 | T_{M}3 |
|---|---|---|---|---|---|---|---|
| Formul. | Buffer | Bulking agent | Polysorbate 20 | pH | (° C.) | (° C.) | (° C.) |
| 1 | 20 mM Cit | 150 mM L-arg | 0.01% | pH 6 | 69.4 | 82.3 | 88.1 |
| 2 | 20 mM Phos | 150 mM L-arg | 0.01% | pH 6 | 69.0 | 82.4 | 88.1 |
| 3 | 20 mM His | 150 mM L-arg | 0.01% | pH 6 | 66.9 | 81.8 | 87.3 |
| 4 | 20 mM His | 150 mM NaCl | 0.01% | pH 6 | 68.4 | 81.3 | 87.0 |

Three thermal transitions were detected upon heating the various formulations of the anti-FGFR2-IIIb antibody. The anti-FGFR2-IIIb antibody started to unfold at 52.7° C. in pH 4 formulation, unfolding temperature (Tm) increased with the increasing pH and reached a maximum Tm1 at around 70° C. at pH 6 and pH 7, and the Tm dropped slightly at pH 8.

Physical Stability

Since protein pharmaceuticals are prone to aggregate upon exposure to shear stresses, physical stability studies were performed to evaluate the effects that excipients have on the stability of the anti-FGFR2-IIIb antibody against multiple freeze/thaw cycles and vigorous agitation. The stability was assessed by visual observation, A350 light scattering, and SE-HPLC for insoluble and soluble aggregates. The antibody concentration in the samples was 20 mg/mL.

Agitation stress was exerted on the samples by placing samples vials horizontally on an orbital shaker and shaking samples at 500 RPM for 77 hours at room temperature. To assess the effect of polysorbate 20 on preventing aggregation formation, a head-to-head comparison was performed of the arginine formulation with 0%, 0.01%, 0.05% and 0.10% polysorbate 20. As shown in Table 8 and FIG. 12, no apparent increase in the soluble aggregates was detected by SE-HPLC in all formulations.

A freeze/thaw study was also performed by freezing samples at 70° C. and thawing at ambient temperature through 5 cycles. As shown in FIG. 13, the only apparent change was observed in the soluble aggregate in the histidine/NaCl formulation, which increased with the increasing freeze/thaw cycles. Adding 0.01% polysorbate 20 to the formulation enhanced the stability of the anti-FGFR2-IIIb antibody against shaking stress.

TABLE 8
Turbidity of Shear Stressed Samples Determined By A350 Reading

| | | | | Shaking at | | Freeze/Thaw | |
|---|---|---|---|---|---|---|---|
| | | | | 500 RPM | | (−70° C. to RT) | |
| ID | Buffer | Bulking agent | PS20 | Initial | 77 hr | Initial | 5 cycles |
| 1 | 20 mM Cit | 150 mM L-arg | 0.01% | 0.081 | 0.077 | 0.077 | 0.073 |
| 2 | 20 mM Phos | 150 mM L-arg | 0.01% | 0.078 | 0.074 | 0.070 | 0.074 |
| 3 | 20 mM His | 150 mM L-arg | 0.01% | 0.086 | 0.087 | 0.077 | 0.082 |
| 4 | 20 mM His | 150 mM NaCl | 0.01% | 0.084 | 0.089 | 0.079 | 0.087 |
| 5 | 20 mM His | 150 mM L-arg | 0.00% | 0.082 | 0.157 | 0.077 | 0.081 |
| 6 | 20 mM His | 150 mM L-arg | 0.05% | 0.087 | 0.086 | ND | 0.082 |
| 7 | 20 mM His | 150 mM L-arg | 0.10% | 0.101 | 0.090 | ND | 0.081 |

Isothermal Stability

The impact of buffer species and bulking agents on the formation of aggregates, clips, and charge variants were assessed by monitoring the stability of the anti-FGFR2-IIIb antibody under accelerated storage conditions at 40° C., 25° C. and 5° C. The changes in aggregates and clips were monitored with SE-HPLC. The following formulations were tested: 20 mM Citrate, 150 mM L-arginine, and 0.01% Polysorbate 20; 20 mM Phosphate, 150 mM L-arginine, and 0.01% Polysorbate 20; 20 mM Histidine, 150 mM L-arginine, and 0.01% Polysorbate 20; 20 mM Histidine, 150 mM NaCl, and 0.01% Polysorbate 20; and 20 mM Histidine, 150 mM L-arginine, no Polysorbate 20.

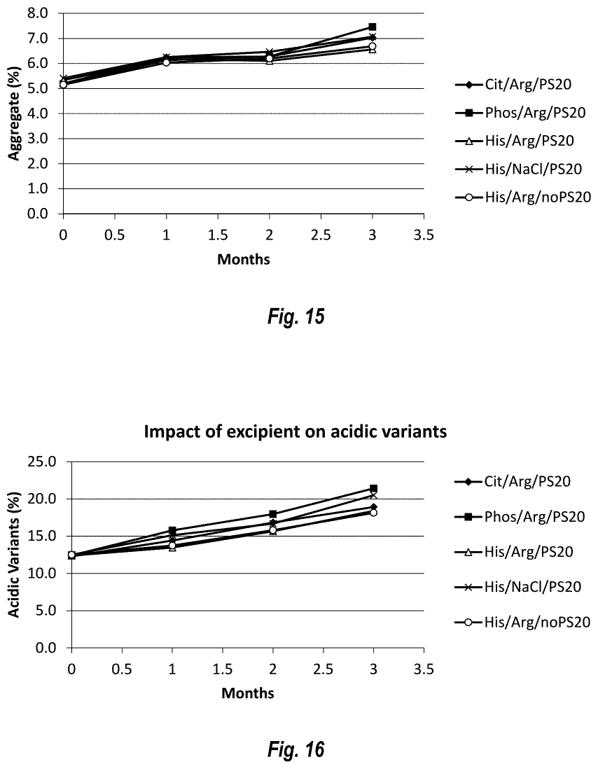

As shown in FIG. 14, aggregates formed at higher rates in the citrate and phosphate formulations at 40° C. Insignificant fragmentation was observed in all formulation after 1 month storage at 40° C. All formulations showed the same increase in aggregation after 3 months storage at 25° C. (FIG. 15).

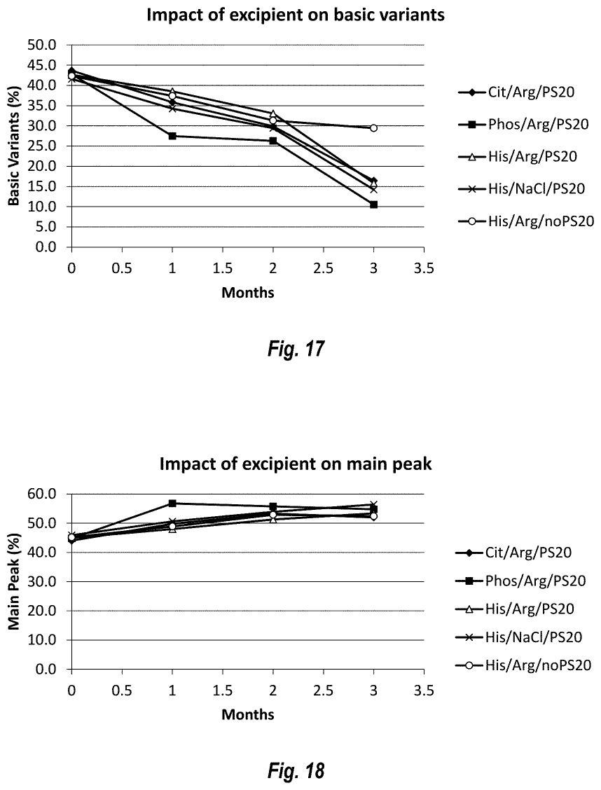

Formulation excipients did not impact the charge profile of the anti-FGFR2-Bib antibody upon storage. As shown in FIG. 16, there was an increase of acidic variants in all formulations. FIG. 17 shows that there was a decrease of basic variants in all formulations over time, and FIG. 18 shows the impact of excipients on the main peak over time.

Based on the antibody stability studies, L-histidine did not have a significant impact on antibody stability among the buffer species evaluated. Sodium citrate and sodium phosphate had an increase in aggregates after 1 month at 40° C. Thus, L-histidine was more stable than sodium citrate or sodium phosphate; aggregates increased at a faster rate in the citrate and phosphate formulations.

Among the bulking agents evaluated, NaCl produced aggregate formation induced by exposure vigorous shaking and freeze/thaw process. The most stable antibody profile was obtained with the L-arginine formulation.

### Example 7: pH Evaluation Study

The effect of pH on the chemical and physical stabilities of the formulations was tested. The following formulations were tested at pH 5.5, 6.0, and 6.5: (a) 20 mM histidine, 150 mM arginine, 0.01% polysorbate 20, and (b) 20 mM histidine, 270 mM sucrose, 0.01% polysorbate 20 (Table 9). The formulations were adjusted to 20 mg/mL and filled into glass vials.

TABLE 9
Formulations evaluated to narrow pH range

| | | | Conc. |
|---|---|---|---|
| ID | Formulation | pH | (mg/ml) |
| 1 | 20 mM Histidine, 150 mM Arginine, 0.01% PS20 | 5.5 | 20 |
| 2 | 20 mM Histidine, 150 mM Arginine, 0.01% PS20 | 5.7 | 20 |
| 3 | 20 mM Histidine, 150 mM Arginine, 0.01% PS20 | 6.0 | 20 |
| 4 | 20 mM Histidine, 150 mM Arginine, 0.01% PS20 | 6.3 | 20 |
| 5 | 20 mM Histidine, 150 mM Arginine, 0.01% PS20 | 6.5 | 20 |
| 6 | 20 mM Histidine, 270 mM Sucrose, 0.01% PS20 | 5.5 | 20 |
| 7 | 20 mM Histidine, 270 mM Sucrose, 0.01% PS20 | 5.7 | 20 |
| 8 | 20 mM Histidine, 270 mM Sucrose, 0.01% PS20 | 6.0 | 20 |
| 9 | 20 mM Histidine, 270 mM Sucrose, 0.01% PS20 | 6.3 | 20 |
| 10 | 20 mM Histidine, 270 mM Sucrose, 0.01% PS20 | 6.5 | 20 |

Effect of pH on Physical Stability

Stability against multiple freeze/thaw cycles and vigorous agitation was evaluated. The stability was assessed by visual observation, A350 light scattering, and SE-HPLC for insoluble and soluble aggregates.

I. Mechanical Agitation

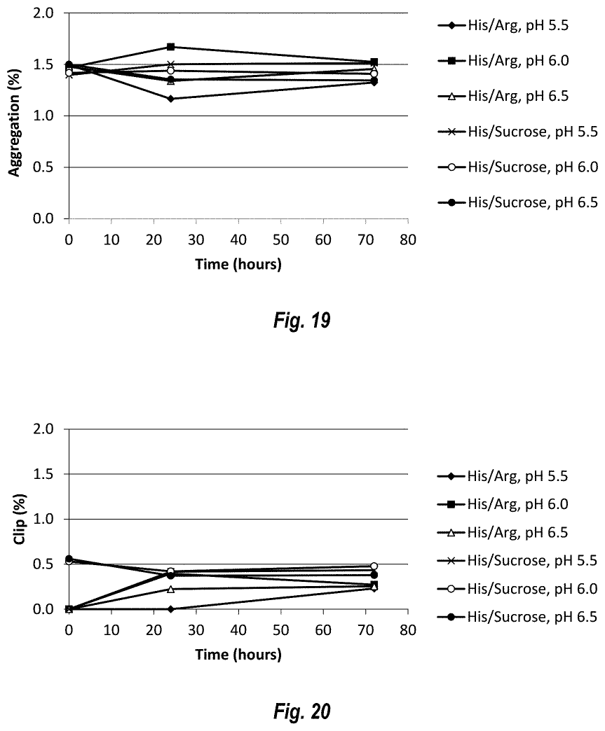

Agitation stress was exerted on the samples by placing samples vials horizontally on an orbital shaker and shaking samples at 500 RPM for 72 hours at room temperature. No apparent increase in the soluble aggregates was detected by SE-HPLC in all formulation. FIG. 19 shows the impact of mechanical stress on aggregation for histidine/arginine and histidine/sucrose formulations between pH 5.5 and 6.5 over time (pH 5.5, 6.0 and 6.5). FIG. 19 shows the impact of mechanical stress on clips for histidine/arginine and histidine/sucrose formulations between pH 5.5 and 6.5. There was no significant change in concentration or light scattering at A350 nm after 72 hours of mechanical agitation (shown in Table 10).

TABLE 10
Effect of Mechanical Agitation on Concentration
and Light Scattering at A350 nm

| | A350 | | Concentration | |
|---|---|---|---|---|
| | 0 hours | 72 hours | 0 hours | 72 hours |
| His/Arg, pH 5.5 | 0.082 | 0.081 | 19.3 | 19.8 |
| His/Arg, pH 6.0 | 0.080 | 0.083 | 19.5 | 19.8 |
| His/Arg, pH 6.5 | 0.084 | 0.091 | 19.7 | 19.7 |
| His/Sucrose, pH 5.5 | 0.085 | 0.085 | 19.6 | 20.0 |
| His/Sucrose, pH 6.0 | 0.084 | 0.088 | 19.5 | 20.1 |
| His/Sucrose, pH 6.5 | 0.089 | 0.118 | 19.8 | 20.2 |

II. Freeze Thaw Stability

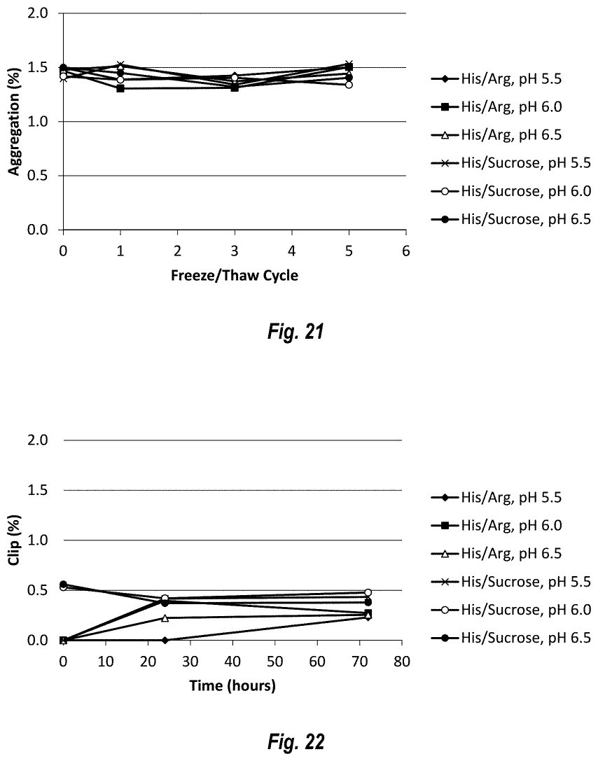

A freeze/thaw study was performed by freezing samples at 70° C. and thawing at ambient temperature through 5 cycles. FIGS. 21 and 22 show the impact of freeze thaw on aggregation and clips for histidine/arginine and histidine/sucrose formulations between pH 5.5 and 6.5 In FIG. 21, there was no apparent increase in the soluble aggregates detected by SE-HPLC in any of the histidine/arginine and histidine/sucrose formulations between pH 5.5 and 6.5. In FIG. 22, no apparent increase in clips was detected by SE-HPLC in any of the formulations.

TABLE 11
Effect of freeze thaw on concentration
and light scattering at A350 nm.

| | A350 | | Concentration | |
|---|---|---|---|---|
| | 0 | Cycle 5 | 0 | Cycle 5 |
| Histidine/Arginine, pH 5.5 | 0.082 | 0.087 | 19.3 | 19.5 |
| Histidine/Arginine, pH 6.0 | 0.080 | 0.089 | 19.5 | 20.1 |
| Histidine/Arginine, pH 6.5 | 0.084 | 0.084 | 19.7 | 19.9 |
| Histidine/Sucrose, pH 5.5 | 0.085 | 0.085 | 19.6 | 19.8 |
| Histidine/Sucrose, pH 6.0 | 0.084 | 0.091 | 19.5 | 20.2 |
| Histidine/Sucrose, pH 6.5 | 0.089 | 0.090 | 19.8 | 20.1 |

Concentration and light scattering remained unchanged after freezing and thawing.

I. Effect of pH on Isothermal Stability

The impact of buffer pH on the formation of aggregates, clips, and charge variants was assessed by monitoring the sample stability under accelerated storage conditions at 40° C., 25° C., and real time stability at 5° C. The following formulations were tested: (A) 20 mM histidine, 150 mM arginine, 0.01% polysorbate 20 (“histidine/arginine formulation”), and (B) 20 mM histidine, 150 mM sucrose, 0.01% polysorbate 20 (“histidine/sucrose formulation”).

Aggregation and clip formation increased with pH after 1 month at 40° C. and 3 months at 25° C. in the histidine/arginine and histidine/sucrose formulations. The histidine/sucrose formulation showing a slightly lower rate of aggregation. Aggregation remained unchanged after 6 months at 5° C., and clip formation increased about 1% in both histidine/arginine and histidine/sucrose formulations.

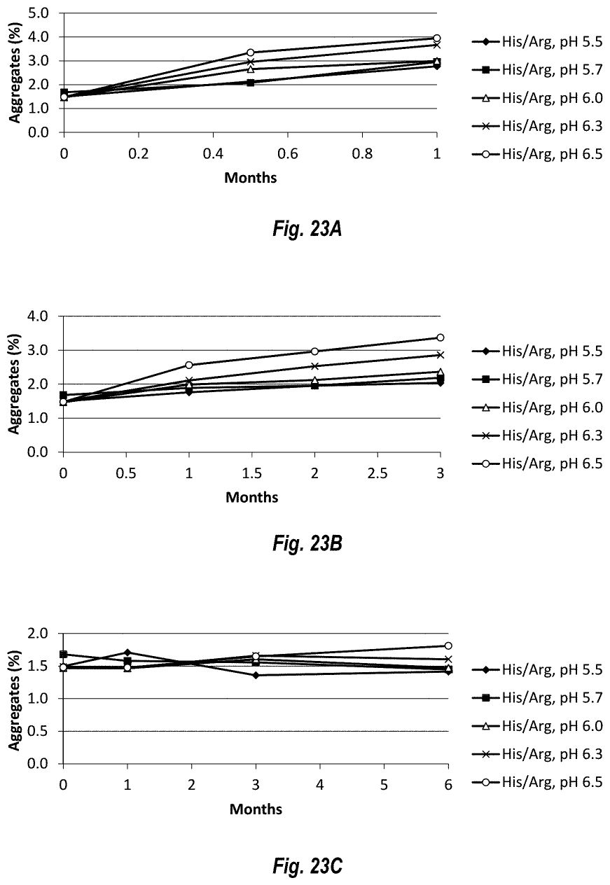

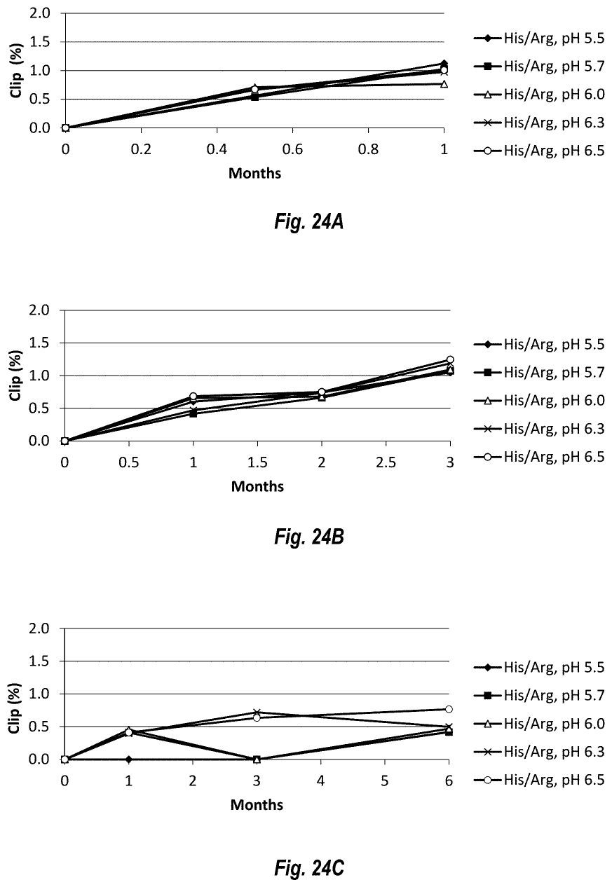

For example, FIGS. 23A-C show the formation of aggregates in the histidine/arginine formulation for various pH conditions measured over (A) 1 month at 40° C., (B) 3 months at 25° C., and (C) 6 months at 5° C. FIGS. 24A-C show clip formation in the histidine/arginine formulation for various pH conditions (pH 5.5-6.5) measured over (A) 1 month at 40° C., (B) 3 months at 25° C., and (C) 6 months at 5° C.

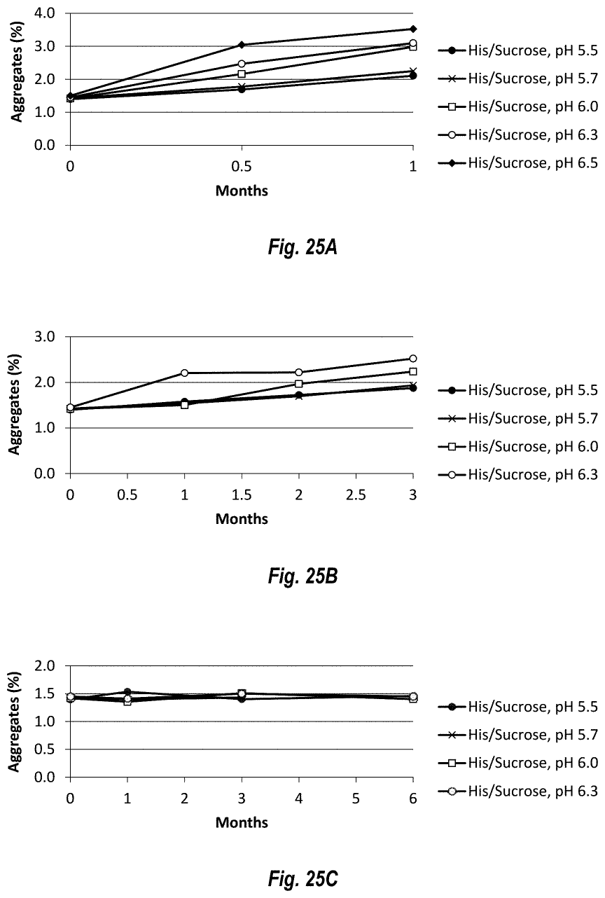

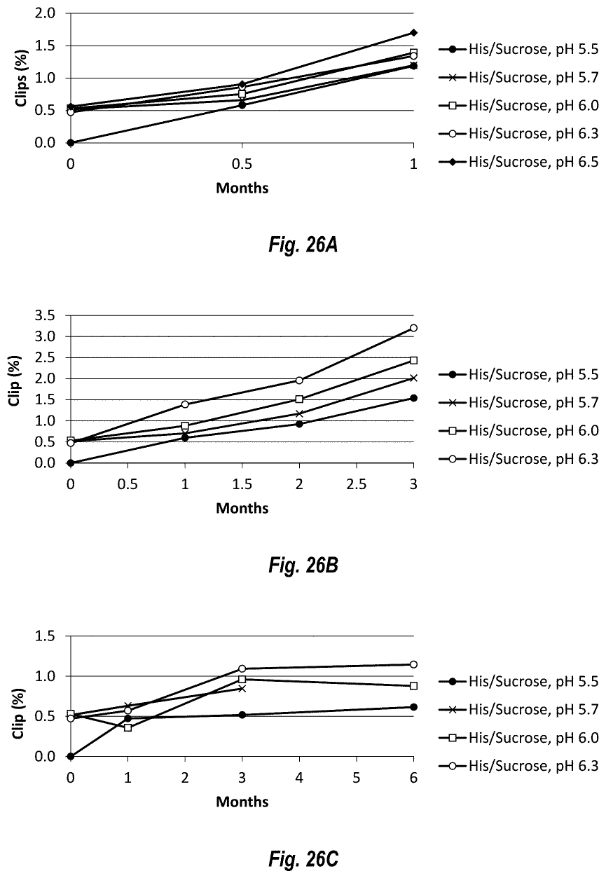

FIGS. 25A-C show aggregate formation in the histidine/sucrose formulation for various pH conditions measured over (A) 1 month at 40° C., (B) 3 months at 25° C., and (C) 6 months at 5° C. FIGS. 26A-C shows the clip formation of the antibody in the histidine/sucrose formulation for various pH conditions (pH 5.5-6.5) measured over (A) 1 month at 40° C., (B) 3 months at 25° C., and (C) 6 months at 5° C.

Charge isoforms also remained unchanged after 6 months at 5° C. For example, FIGS. 27A-C show the acidic variants in the histidine/sucrose formulations increased after (A) 1 month at 40° C., (B) 3 months at 25° C., and (C) 6 months at 5° C.

In addition, FIGS. 28A-C show effect of pH between pH 5.5 and 6.5 on acidic variant formation in a histidine/arginine formulation comprising 20 mM histidine, 150 mM arginine, and 0.01% polysorbate 20. FIG. 28A shows acidic variant formation, indicating that, at pH 5.5-6.0, after 1 month at 40° C., % acidic variants remained generally below 40%. FIG. 28B shows acidic variant formation, indicating that, at pH 5.5-6.0, after 3 months at 25° C., % acidic variants remained generally below 30.0%. FIG. 28C shows acidic variant formation, indicating that, at all pHs, after 6 months at 5° C., % acidic variants remained generally below 25%. FIGS. 29A-C show effect of pH between pH 5.5 and 6.5 on basic variant formation in a histidine/sucrose formulation comprising 20 mM histidine, 270 mM sucrose, and 0.01% polysorbate 20. FIG. 29A shows basic variant formation, indicating that, at pH 5.5-6.0, after 1 month at 40° C., % basic variants remained mostly between 10% and 20%. FIG. 29B shows basic variant formation, indicating that, at pH 5.5-6.0, after 3 months at 25° C., % basic variants remained generally between 20% and 30%. FIG. 29C shows basic variant formation, indicating that, at all pHs, after 6 months at 5° C., % basic variants remained generally between 25% and 30%.

FIGS. 30A-C show effect of pH between pH 5.5 and 6.5 on basic variant formation in a histidine/arginine formulation comprising 20 mM histidine, 150 mM arginine, and 0.01% polysorbate 20. FIG. 30A shows basic variant formation, indicating that, at pH 5.5-6.0, after 1 month at 40° C., % basic variants remained mostly between 10% and 20%. FIG. 30B shows basic variant formation, indicating that, at pH 5.5-6.0, after 3 months at 25° C., % basic variants remained generally between 15% and 25%. FIG. 30C shows basic variant formation, indicating that, at all pHs, after 6 months at 5° C., % basic variants remained generally between 25% and 30%.

Both the histidine/arginine and histidine/sucrose formulations maintained acceptable stability between pH 5.5 and pH 6.5. Overall, the histidine/sucrose formulation was more stable than histidine/arginine between pH 5.5 and 6.0.

Based on the data for formulation stability at 3 months at 25° C. and stability at 6 months at 5° C., and data from the other formulation studies in the Examples above, a liquid formulation was developed containing 20 mg/mL anti-FGFR2-IIIb antibody, 20 mM L-histidine, 270 mM sucrose, and 0.01% polysorbate 20 at a pH of 6.0. A second formulation was also developed containing 20 mg/mL anti-FGFR2-IIIb antibody, 20 mM L-histidine, 150 mM L-arginine, 0.01% polysorbate 20 at a pH 5.7.

The invention is not to be limited in scope by the specific embodiments described herein. Indeed, various modifications provided herein in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes.

Other embodiments are within the claims that follow.

### TABLE OF SEQUENCES

The sequence table below provides certain sequences discussed herein. All polypeptide and antibody sequences are shown without leader sequences, unless otherwise indicated.

Table of Sequences and Descriptions

| SEQ | | |
|---|---|---|
| ID | | |
| NO | Description | Sequence |
| 1 | Mature | RPSFSLVED TTLEPEEPPT KYQISQPEVY VAAPGESLEV RCLLKDAAVI |
| | human | SWTKDGVHLG PNNRTVLIGE YLQIKGATPR DSGLYACTAS RTVDSETWYF |
| | FGFR2-IIIb | MVNVTDAISS GDDEDDTDGA EDFVSENSNN KRAPYWTNTE KMEKRLHAVP |
| | | AANTVKFRCP AGGNPMPTMR WLKNGKEFKQ EHRIGGYKVR NQHWSLIMES |
| | | VVPSDKGNYT CVVENEYGSI NHTYHLDVVE RSPHRPILQA GLPANASTVV |
| | | GGDVEFVCKV YSDAQPHIQW IKHVEKNGSK YGPDGLPYLK VLKHSGINSS |
| | | NAEVLALFNV TEADAGEYIC KVSNYIGQAN QSAWLTVLPK QQAPGREKEI |
| | | TASPDYLEIA IYCIGVFLIA CMVVTVILCR MKNTTKKPDF SSQPAVHKLT |
| | | KRIPLRRQVT VSAESSSSMN SNTPLVRITT RLSSTADTPM LAGVSEYELP |
| | | EDPKWEFPRD KLTLGKPLGE GCFGQVVMAE AVGIDKDKPK EAVTVAVKML |
| | | KDDATEKDLS DLVSEMEMMK MIGKHKNIIN LLGACTQDGP LYVIVEYASK |
| | | GNLREYLRAR RPPGMEYSYD INRVPEEQMT FKDLVSCTYQ LARGMEYLAS |
| | | QKCIHRDLAA RNVLVTENNV MKIADFGLAR DINNIDYYKK TTNGRLPVKW |
| | | MAPEALFDRV YTHQSDVWSF GVLMWEIFTL GGSPYPGIPV EELFKLLKEG |
| | | HRMDKPANCT NELYMMMRDC WHAVPSQRPT FKQLVEDLDR ILTLTTNEEY |
| | | LDLSQPLEQY SPSYPDTRSS CSSGDDSVFS PDPMPYEPCL PQYPHINGSV |
| | | KT |
| 2 | αFGFR2b | QVQLVQSGAE VKKPGSSVKV SCKASGYIFT TYNVHWVRQA PGQGLEWIGS |
| | heavy | IYPDNGDTSY NQNFKGRATI TADKSTSTAY MELSSLRSED TAVYYCARGD |
| | chain; | FAYWGQGTLV TVSSASTKGP SVFPLAPSSK STSGGTAALG CLVKDYFPEP |
| | Asn297 is | VTVSWNSGAL TSGVHTFPAV LQSSGLYSLS SVVTVPSSSL GTQTYICNVN |
| | in bold | HKPSNTKVDK RVEPKSCDKT HTCPPCPAPE LLGGPSVFLF PPKPKDTLMI |
| | and | SRTPEVTCVV VDVSHEDPEV KENWYVDGVE VHNAKTKPRE EQY [symbol] STYRVV |
| | underlined | SVLTVLHQDW LNGKEYKCKV SNKALPAPIE KTISKAKGQP REPQVYTLPP |
| | | SREEMTKNQV SLTCLVKGFY PSDIAVEWES NGQPENNYKT TPPVLDSDGS |
| | | FFLYSKLTVD KSRWQQGNVF SCSVMHEALH NHYTQKSLSL SPGK |
| 3 | αFGFR2b | DIQMTQSPSS LSASVGDRVT ITCKASQGVS NDVAWYQQKP GKAPKLLIYS |
| | light | ASYRYTGVPS RFSGSGSGTD FTFTISSLQP EDIATYYCQQ HSTTPYTFGQ |
| | chain | GTKLEIKRTV AAPSVFIFPP SDEQLKSGTA SVVCLLNNFY PREAKVQWKV |
| | | DNALQSGNSQ ESVTEQDSKD STYSLSSTLT LSKADYEKHK VYACEVTHQG |
| | | LSSPVTKSFN RGEC |
| 4 | αFGFR2b | QVQLVQSGAE VKKPGSSVKV SCKASGYIFT TYNVHWVRQA PGQGLEWIGS |
| | heavy | IYPDNGDTSY NQNFKGRATI TADKSTSTAY MELSSLRSED TAVYYCARGD |
| | chain | FAYWGQGTLV TVSS |
| | variable | |
| | region | |
| 5 | αFGFR2b | DIQMTQSPSS LSASVGDRVT ITCKASQGVS NDVAWYQQKP GKAPKLLIYS |
| | light | ASYRYTGVPS RFSGSGSGTD FTFTISSLQP EDIATYYCQQ HSTTPYTFGQ |
| | chain | GTKLEIK |
| | variable | |
| | region | |
| 6 | αFGFR2b | TYNVH |
| | heavy | |
| | chain (HC) | |
| | HVR1 | |
| 7 | αFGFR2b HC | SIYPDNGDTS YNQNFKG |
| | HVR2 | |
| 8 | αFGFR2b HC | GDFAY |
| | HVR3 | |
| 9 | αFGFR2b | KASQGVSNDV A |
| | light | |
| | chain (LC) | |
| | HVR1 | |
| 10 | αFGFR2b LC | SASYRYT |
| | HVR2 | |
| 11 | αFGFR2b LC | QQHSTTPYT |
| | HVR3 | |
| 12 | αFGFR2b | QVQLVQSGAE VKKPGSSVKV SCKASGYIFT TYNVHWVRQA PGQGLEWIGS |
| | N297Q | IYPDNGDTSY NQNFKGRATI TADKSTSTAY MELSSLRSED TAVYYCARGD |
| | heavy | FAYWGQGTLV TVSSASTKGP SVFPLAPSSK STSGGTAALG CLVKDYFPEP |
| | chain; the | VTVSWNSGAL TSGVHTFPAV LQSSGLYSLS SVVTVPSSSL GTQTYICNVN |
| | N297Q | HKPSNTKVDK RVEPKSCDKT HTCPPCPAPE LLGGPSVFLF PPKPKDTLMI |
| | point | SRTPEVTCVV VDVSHEDPEV KFNWYVDGVE VHNAKTKPRE EQY [symbol] STYRVV |
| | mutation | SVLTVLHQDW LNGKEYKCKV SNKALPAPIE KTISKAKGQP REPQVYTLPP |
| | is bold | SREEMTKNQV SLTCLVKGFY PSDIAVEWES NGQPENNYKT TPPVLDSDGS |
| | and | FFLYSKLTVD KSRWQQGNVF SCSVMHEALH NHYTQKSLSL SPGK |
| | underlined | |
| 13 | Mature | RPSFSLVED TTLEPEEPPT KYQISQPEVY VAAPGESLEV RCLLKDAAVI |
| | human | SWTKDGVHLG PNNRTVLIGE YLQIKGATPR DSGLYACTAS RTVDSETWYF |
| | FGFR2-IIIc | MVNVTDAISS GDDEDDTDGA EDFVSENSNN KRAPYWTNTE KMEKRLHAVP |
| | | AANTVKFRCP AGGNPMPTMR WLKNGKEFKQ EHRIGGYKVR NQHWSLIMES |
| | | VVPSDKGNYT CVVENEYGSI NHTYHLDVVE RSPHRPILQA GLPANASTVV |
| | | GGDVEFVCKV YSDAQPHIQW IKHVEKNGSK YGPDGLPYLK VLKAAGVNTT |
| | | DKEIEVLYIR NVTFEDAGEY TCLAGNSIGI SFHSAWLTVL PAPGREKEIT |
| | | ASPDYLEIAI YCIGVFLIAC MVVTVILCRM KNTTKKPDFS SQPAVHKLTK |
| | | RIPLRRQVTV SAESSSSMNS NTPLVRITTR LSSTADTPML AGVSEYELPE |
| | | DPKWEFPRDK LTLGKPLGEG CFGQVVMAEA VGIDKDKPKE AVTVAVKMLK |
| | | DDATEKDLSD LVSEMEMMKM IGKHKNIINL LGACTQDGPL YVIVEYASKG |
| | | NLREYLRARR PPGMEYSYDI NRVPEEQMTF KDLVSCTYQL ARGMEYLASQ |
| | | KCIHRDLAAR NVLVTENNVM KIADFGLARD INNIDYYKKT |
| | | TNGRLPVKWM APEALFDRVY THQSDVWSFG VLMWEIFTLG GSPYPGIPVE |
| | | ELFKLLKEGH RMDKPANCTN ELYMMMRDCW HAVPSQRPTF KQLVEDLDRI |
| | | LTLTTNEEYL DLSQPLEQYS PSYPDTRSSC SSGDDSVESP DPMPYEPCLP |
| | | QYPHINGSVK T |
| 14 | FGFR2 ECD | RPSFSLVED TTLEPEEPPT KYQISQPEVY VAAPGESLEV RCLLKDAAVI |
| | | SWTKDGVHLG PNNRTVLIGE YLQIKGATPR DSGLYACTAS RTVDSETWYF |
| | | MVNVTDAISS GDDEDDTDGA EDFVSENSNN KRAPYWTNTE KMEKRLHAVP |
| | | AANTVKFRCP AGGNPMPTMR WLKNGKEFKQ EHRIGGYKVR NQHWSLIMES |
| | | VVPSDKGNYT CVVENEYGSI NHTYHLDVVE RSPHRPILQA GLPANASTVV |
| | | GGDVEFVCKV YSDAQPHIQW IKHVEKNGSK YGPDGLPYLK VLKAAGVNTT |
| | | DKEIEVLYIR NVTFEDAGEY TCLAGNSIGI SFHSAWLTVL PAPGREKEIT |
| | | ASPDYLE |
| 15 | Anti-FGFR2 | QVQLKQSGPG LVQPSQSLSI TCTVSGFSLT SFGVHWVRQS PGKGLEWLGV |
| | Gal-FR22 | IWSGGSTDYN ADFRSRLSIS KDNSKSQIFF KMNSLQPDDT IAYCANFYYG |
| | heavy | YDDYVMDYWG QGTSVTVSS |
| | chain | |
| | variable | |
| | region | |
| 16 | Anti-FGFR2 | SFGVH |
| | Gal-FR22 | |
| | heavy | |
| | chain CDR1 | |
| 17 | Anti-FGFR2 | VIWSGGSTDYNADFRS |
| | Gal-FR22 | |
| | heavy | |
| | chain CDR2 | |
| 18 | Anti-FGFR2 | FYYGYDDYVMDY |
| | Gal-FR22 | |
| | heavy | |
| | chain CDR3 | |
| 19 | Anti-FGFR2 | DIQMTQSPSS LSASLGGRVT ITCKASQDIK NYIAWYQHKP GKSPRLLIHY |
| | Gal-FR22 | TSTLQPGVPS RFSGSGSGRD YSFSISNLEP EDIATYYCLQ YDDDLYMFGG |
| | light | GTKLDIK |
| | chain | |
| | variable | |
| | region | |
| 20 | Anti-FGFR2 | KASQDIKNYIA |
| | Gal-FR22 | |
| | light | |
| | chain CDR1 | |
| 21 | Anti-FGFR2 | YTSTLQP |
| | Gal-FR22 | |
| | light | |
| | chain CDR2 | |
| 22 | Anti-FGFR2 | LQYDDLYM |
| | Gal-FR22 | |
| | light | |
| | chain CDR3 | |

1. A pharmaceutical formulation, comprising:
i) 10-30 mg/mL of an anti-Fibroblast Growth Factor Receptor 2 (FGFR2) antibody;
ii) 5-40 mM of a buffer selected from one or more of histidine, citrate, or phosphate;
iii) 130 to 170 mM arginine or 250-290 mM sucrose; and
iv) 0.002% to 0.1% polysorbate 20 or polysorbate 80;
wherein the formulation has a pH of 5.0 to 6.5, and wherein the anti-FGFR2 antibody is selected from:
a) an antibody comprising a heavy chain comprising the sequence of SEQ ID NO: 2 and a light chain comprising the sequence of SEQ ID NO: 3;
b) an antibody comprising a heavy chain comprising a heavy chain (HC) hypervariable region 1 (HVR1) comprising the sequence of SEQ ID NO: 6, an HC HVR2 comprising the sequence of SEQ ID NO: 7, and an HC HVR3 comprising the sequence of SEQ ID NO: 8, and a light chain comprising a light chain (LC) HVR1 comprising the sequence of SEQ ID NO: 9, a LC HVR2 comprising the sequence of SEQ ID NO: 10, and a LC HVR3 comprising the sequence of SEQ ID NO: 11; and
c) an antibody comprising a heavy chain comprising a variable region sequence comprising the sequence of SEQ ID NO: 4 and a light chain comprising a light chain variable region sequence comprising the sequence of SEQ ID NO: 5.

2. The pharmaceutical formulation of claim 1, wherein the formulation has one or more of the following properties:
(a) is a ready-to-use, liquid formulation;
(b) is not lyophilized prior to administration to a patient;
(c) is contained within a single-use vial;
(d) protein aggregation in the formulation increases by no more than 2.0% after 6 months storage at 5° C.;
(e) protein aggregation in the formulation increases by no more than 2.0% or 2.5% after 3 months storage at 25° C.;
(f) protein aggregation in the formulation increases by no more than 7.0% after 3 months storage at 40° C.;
(g) charged protein variants in the formulation do not change by more than 5% after 6 months of storage at 5° C.;
(h) protein aggregation in the formulation increases by no more than 2.0% after 5 freeze-thaw cycles at −70° C.;
(i) protein aggregation in the formulation increases by no more than 2.0% after 72 hours of mechanical stress at 500 rpm;
(j) is diluted in saline solution prior to intravenous administration;
(k) is administered intravenously (e.g. by intravenous infusion); and
(l) is isotonic with human plasma.

3. The pharmaceutical formulation of claim 1 or 2, wherein the formulation comprises 10-15 mg/mL, 15-20 mg/mL, 20-25 mg/mL, 18-22 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, or 25 mg/mL of the anti-FGFR2 antibody.

4. The pharmaceutical formulation of any one of claims 1-3, wherein the formulation comprises 10-40 mM, 10-30 mM, 15-25 mM, 10-20 mM, 20-30 mM, 18-22 mM, 10 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, or 30 mM of the buffer.

5. The pharmaceutical formulation of claim 4, wherein the buffer is a histidine buffer.

6. The pharmaceutical formulation of any one of claims 1-5, wherein the formulation comprises 130-150 mM, 150-170 mM, 140-160 mM, 130 mM, 135 mM, 140 mM, 145 mM, 150 mM, 155 mM, 160 mM, 165 mM, or 170 mM arginine.

7. The pharmaceutical formulation of claim 6, wherein the formulation does not comprise sucrose.

8. The pharmaceutical formulation of claim 6 or 7, wherein the pH of the formulation is 5.0-7.0, 5.0-6.0, 5.5-6.0, 5.5-6.5, 5.5-5.9, 5.6-5.8, 5.0, 5.1, 5.2, 5.3, 5.4, 5.5, 5.6, 5.7, 5.8, 5.9, 6.0, 6.1, 6.2, 6.3, 6.4, or 6.5.

9. The pharmaceutical formulation of claim 8, wherein the pH of the formulation is 5.5-5.9, 5.6-5.8, 5.6, 5.7, 5.8, 5.9, or 6.0.

10. The pharmaceutical formulation of any one of claims 1-5, wherein the formulation comprises 260-280 mM, 250 mM, 260 mM, 270 mM, 280 mM, 290 mM sucrose.

11. The pharmaceutical formulation of claim 8, wherein the formulation does not comprise arginine.

12. The pharmaceutical formulation of claim 10 or 11, wherein the pH of the formulation is 5.0-7.0, 5.0-6.0, 5.5-6.0, 5.5-5.9, 5.6-5.8, 5.0, 5.1, 5.2, 5.3, 5.4, 5.5, 5.6, 5.7, 5.8, 5.9, 6.0, 6.1, 6.2, 6.3, 6.4, or 6.5.

13. The pharmaceutical formulation of claim 12, wherein the pH of the formulation is 5.8-6.2, 5.9-6.1, 5.9, 6.0, or 6.1.

14. The pharmaceutical formulation of any one of claims 1-13, wherein the formulation comprises 0.01-0.1%, 0.005-0.05%, 0.002%, 0.003%. 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, or 0.1% polysorbate 20 or 80.

15. The pharmaceutical formulation of claim 14, wherein the formulation comprises 0.01-0.1%, 0.005-0.05%, 0.002%, 0.003%. 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, or 0.1% polysorbate 20.

16. The pharmaceutical formulation of any one of claims 1-15, wherein the formulation consists essentially of anti-FGFR2 antibody; citrate, phosphate, or histidine buffer; arginine or sucrose; and polysorbate 20 or 80.

17. The pharmaceutical formulation of claim 16, wherein the formulation consists essentially of anti-FGFR2 antibody, histidine, arginine or sucrose, and polysorbate 20.

18. A pharmaceutical formulation of an anti-Fibroblast Growth Factor Receptor 2 (FGFR2) antibody, wherein the formulation is a liquid formulation that has not been lyophilized prior to use, comprising:
i) 10-25 mg/mL of the anti-FGFR2 antibody;
ii) 10-30 mM histidine buffer;
iii) 140-160 mM arginine; and
iv) 0.005% to 0.05% polysorbate 20;
wherein the formulation has a pH of 5.6 to 5.8.

19. A pharmaceutical formulation of an anti-Fibroblast Growth Factor Receptor 2 (FGFR2) antibody, wherein the formulation is a liquid formulation that has not been lyophilized prior to use, comprising:
i) 10-25 mg/mL of the anti-FGFR2 antibody;
ii) 20 mM histidine buffer;
iii) 150 mM arginine; and
iv) 0.01% polysorbate 20;
wherein the formulation has a pH of 5.7.

20. A pharmaceutical formulation of an anti-Fibroblast Growth Factor Receptor 2 (FGFR2) antibody, comprising:
i) 20 mg/mL of the anti-FGFR2 antibody;
ii) 20 mM L-histidine buffer;
iii) 150 mM L-arginine;
iv) 0.01% polysorbate 20,
wherein the formulation has a pH of 5.7 and is a liquid formulation that has not been lyophilized prior to use.

21. The pharmaceutical formulation of claim 18, 19, or 20, wherein the formulation consists essentially of the anti-FGFR2 antibody, histidine, arginine, and polysorbate 20.

22. A pharmaceutical formulation of an anti-Fibroblast Growth Factor Receptor 2 (FGFR2) antibody, wherein the formulation is a liquid formulation that has not been lyophilized prior to use, comprising:
i) 10-25 mg/mL of the anti-FGFR2 antibody;
ii) 10-30 mM histidine buffer;
iii) 260-280 mM sucrose; and
iv) 0.005% to 0.05% polysorbate 20;
wherein the formulation has a pH of 5.8 to 6.2.

23. A pharmaceutical formulation of an anti-Fibroblast Growth Factor Receptor 2 (FGFR2) antibody, wherein the formulation is a liquid formulation that has not been lyophilized prior to use, comprising:
i) 10-25 mg/mL of the anti-FGFR2 antibody;
ii) 20 mM histidine buffer;
iii) 270 mM sucrose; and
iv) 0.01% polysorbate 20;
wherein the formulation has a pH of 6.0.

24. A pharmaceutical formulation of an anti-Fibroblast Growth Factor Receptor 2 (FGFR2) antibody, comprising:
i) 20 mg/mL of the anti-FGFR2 antibody;
ii) 20 mM L-histidine buffer;
iii) 270 mM sucrose; and
iv) 0.01% polysorbate 20,
wherein the formulation has a pH of 6.0 and is a liquid formulation that has not been lyophilized prior to use.

25. The pharmaceutical formulation of claim 22, 23, or 24, wherein the formulation consists essentially of the anti-FGFR2 antibody, histidine, sucrose, and polysorbate 20.

26. The pharmaceutical formulation of any one of claims 18-25, wherein the anti-FGFR2 antibody is selected from:
a) an antibody comprising a heavy chain comprising the sequence of SEQ ID NO: 2 and a light chain comprising the sequence of SEQ ID NO: 3;
b) an antibody comprising a heavy chain comprising a heavy chain (HC) hypervariable region 1 (HVR1) comprising the sequence of SEQ ID NO: 6, an HC HVR2 comprising the sequence of SEQ ID NO: 7, and an HC HVR3 comprising the sequence of SEQ ID NO: 8, and a light chain comprising a light chain (LC) HVR1 comprising the sequence of SEQ ID NO: 9, a LC HVR2 comprising the sequence of SEQ ID NO: 10, and a LC HVR3 comprising the sequence of SEQ ID NO: 11; and
c) an antibody comprising a heavy chain comprising a variable region sequence comprising the sequence of SEQ ID NO: 4 and a light chain comprising a light chain variable region sequence comprising the sequence of SEQ ID NO: 5.

27. The pharmaceutical formulation of any one of claims 18-26, wherein the formulation has one or more of the following properties:
(a) is contained within a single-use vial;
(b) protein aggregation in the formulation increases by no more than 2.0% after 6 months storage at 5° C.;
(c) protein aggregation in the formulation increases by no more than 2.0% or 2.5% after 3 months storage at 25° C.;
(d) protein aggregation in the formulation increases by no more than 7.0% after 3 months storage at 40° C.;
(e) charged protein variants in the formulation do not change by more than 5% after 6 months of storage at 5° C.;
(f) protein aggregation in the formulation increases by no more than 2.0% after 5 freeze-thaw cycles at −70° C.;
(g) protein aggregation in the formulation increases by no more than 2.0% after 72 hours of mechanical stress at 500 rpm;
(h) is diluted in saline solution prior to intravenous administration;
(i) is administered intravenously (e.g. by IV infusion); and
(j) is isotonic with human plasma.

28. The pharmaceutical formulation of any one of claim 18-19, 21-23, or 25-27, wherein the formulation comprises 10-15 mg/mL, 15-20 mg/mL, 20-25 mg/mL, 18-22 mg/mL, 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 21 mg/mL, 22 mg/mL, 23 mg/mL, 24 mg/mL, or 25 mg/mL of the anti-FGFR2 antibody.

29. The pharmaceutical formulation of any one of claims 1-28, wherein the formulation does not comprise one or more of the following: sugars other than sucrose, sugar alcohols, protein species other than anti-FGFR2 antibody, surfactants other than polysorbate 20 or polysorbate 80, amino acids other than arginine and histidine, Cu²⁺, Mg²⁺, and Mn²⁺.

30. The pharmaceutical formulation of any one of claims 1-29, wherein the formulation is contained within single-use vials.

31. A method of treating a solid tumor in a patient in need thereof, comprising administering an effective amount of the formulation of any one of claims 1-30 to the patient.

32. The method of claim 31, wherein the formulation is administered intravenously to the patient (e.g., by intravenous infusion).

33. The pharmaceutical formulation or method of any one of claims 1-32, wherein the anti-FGFR2 antibody is afucosylated.

34. The pharmaceutical formulation or method of any one of claims 1-32, wherein the anti-FGFR2 antibody is an antigen binding fragment, such as an Fv, single-chain Fv (scFv), Fab, Fab′, or (Fab′)₂.

35. The pharmaceutical formulation or method of any one of claims 1-34, wherein the antibody is chimeric, humanized, or human.

36. The pharmaceutical formulation or method of any one of claims 1-35, wherein the antibody is bispecific, multispecific, or conjugated.


SEQUENCE LISTING (22 sequences)

SEQ ID NO: 1 - protein, 801 aa, Homo sapiens
RPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 2 - protein, 444 aa, Artificial Sequence
  alpha-FGFR2b heavy chain
QVQLVQSGAEVKKPGSSVKVSCKASGYIFTTYNVHWVRQAPGQGLEWIGSIYPDNGDTSYNQNFKGRATITADKSTSTAYMELSSLRSEDTAVYYCARGDFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 214 aa, Artificial Sequence
  alpha-FGFR2b light chain
DIQMTQSPSSLSASVGDRVTITCKASQGVSNDVAWYQQKPGKAPKLLIYSASYRYTGVPSRFSGSGSGTDFTFTISSLQPEDIATYYCQQHSTTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 114 aa, Artificial Sequence [sequence omitted]

SEQ ID NO: 5 - protein, 107 aa, Artificial Sequence [sequence omitted]

SEQ ID NO: 6 - protein, 5 aa, Artificial Sequence
  alpha-FGFR2b heavy chain (HC) HVR1
TYNVH

SEQ ID NO: 7 - protein, 17 aa, Artificial Sequence
  alpha-FGFR2b HC HVR2
SIYPDNGDTSYNQNFKG

SEQ ID NO: 8 - protein, 5 aa, Artificial Sequence
  alpha-FGFR2b HC HVR3
GDFAY

SEQ ID NO: 9 - protein, 11 aa, Artificial Sequence
  alpha-FGFR2b light chain (LC) HVR1
KASQGVSNDVA

SEQ ID NO: 10 - protein, 7 aa, Artificial Sequence
  alpha-FGFR2b LC HVR2
SASYRYT

SEQ ID NO: 11 - protein, 9 aa, Artificial Sequence
  alpha-FGFR2b LC HVR3
QQHSTTPYT

SEQ ID NO: 12 - protein, 444 aa, Artificial Sequence
  alpha-FGFR2b N297Q heavy chain
QVQLVQSGAEVKKPGSSVKVSCKASGYIFTTYNVHWVRQAPGQGLEWIGSIYPDNGDTSYNQNFKGRATITADKSTSTAYMELSSLRSEDTAVYYCARGDFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 800 aa, Homo sapiens
RPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 14 - protein, 356 aa, Artificial Sequence
  FGFR2 ECD
RPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLE

SEQ ID NO: 15 - protein, 119 aa, Artificial Sequence
  Anti-FGFR2 Gal-FR22 heavy chain variable region
QVQLKQSGPGLVQPSQSLSITCTVSGFSLTSFGVHWVRQSPGKGLEWLGVIWSGGSTDYNADFRSRLSISKDNSKSQIFFKMNSLQPDDTIAYCANFYYGYDDYVMDYWGQGTSVTVSS

SEQ ID NO: 16 - protein, 5 aa, Artificial Sequence
  Anti-FGFR2 Gal-FR22 heavy chain CDR1
SFGVH

SEQ ID NO: 17 - protein, 16 aa, Artificial Sequence
  Anti-FGFR2 Gal-FR22 heavy chain CDR2
VIWSGGSTDYNADFRS

SEQ ID NO: 18 - protein, 12 aa, Artificial Sequence
  Anti-FGFR2 Gal-FR22 heavy chain CDR3
FYYGYDDYVMDY

SEQ ID NO: 19 - protein, 107 aa, Artificial Sequence
  Anti-FGFR2 Gal-FR22 light chain variable region
DIQMTQSPSSLSASLGGRVTITCKASQDIKNYIAWYQHKPGKSPRLLIHYTSTLQPGVPSRFSGSGSGRDYSFSISNLEPEDIATYYCLQYDDDLYMFGGGTKLDIK

SEQ ID NO: 20 - protein, 11 aa, Artificial Sequence
  Anti-FGFR2 Gal-FR22 light chain CDR1
KASQDIKNYIA

SEQ ID NO: 21 - protein, 7 aa, Artificial Sequence
  Anti-FGFR2 Gal-FR22 light chain CDR2
YTSTLQP

SEQ ID NO: 22 - protein, 8 aa, Artificial Sequence
  Anti-FGFR2 Gal-FR22 light chain CDR3
LQYDDLYM